(19) United States
(12) Patent Application Publication
(10) Pub. No.: US 2006/0222665 A1
(43) Pub. Date: Oct. 5, 2006

(54) Virus vaccine

(75) Inventor: Michael Schreiber, Hamburg (DE)

Correspondence Address: NIXON & VANDERHYE, PC, 901 NORTH GLEBE ROAD, 11TH FLOOR, ARLINGTON, VA, 22203, US

(73) Assignee: Strathmann AG & Co., Hamburg (DE)

(21) Appl. No.: 11/271,889

(22) Filed: Nov. 14, 2005

Related U.S. Application Data
(62) Division of application No. 09/913,159, filed on Aug. 10, 2001, now abandoned.

(30) Foreign Application Priority Data
Feb. 12, 1999 (DE) 199 07 485.2

(51) Int. Cl. A61K 48/00; A61K 39/21

(52) U.S. Cl. 424/208.1; 514/44

(57) ABSTRACT

The present invention relates to a pharmaceutical composition or a vaccine which comprises a mixture of viral protein molecules which are sequence variants of a single viral protein or of part of same. The invention furthermore relates inter alia to a DNA vaccine which codes for a mixture of structurally different virus proteins, the vaccine containing a mixture of sequence variants of a viral DNA molecule or of part of same which code for sequence variants of a viral protein or part. According to a preferred version of the invention, the viral proteins are sequence variants of the GP120 protein of the human immunodeficiency virus (HIV) which differ from each another in their amino acid sequence in the region of the V2 loop and/or of the V3 loop. The invention furthermore relates to the preparation of the virus vaccines including the intermediate stages or constructs, preparation processes and uses connected with them.

The present application is a divisional of Ser. No. 09/913,159, filed Aug. 10, 2001, which is a 371 U.S. national phase of PCT/EP99/09759, filed Dec. 3, 1999, which designated the U.S. and claims benefit of DE 199 07 485.2, filed Feb. 12, 1999, the entire contents of each of which application is hereby incorporated by reference.

The present invention relates to a pharmaceutical composition or a vaccine which comprises a mixture of viral protein molecules which are sequence variants of a single viral protein or of part of same. The invention furthermore relates inter alia to a DNA vaccine which codes for a mixture of structurally different virus proteins, the vaccine containing a mixture of sequence variants of a viral DNA molecule or of part of same, which code for sequence variants of a viral protein or part. According to a preferred version of the invention, the viral proteins are sequence variants of the GP120 protein of the human immunodeficiency virus (HIV) which differ from each other in their amino acid sequence in the region of the V2 loop and/or of the V3 loop, preferably both the V2 and the V3 loop. The invention furthermore relates to the preparation of the virus vaccines including the intermediate stages or constructs, preparation processes and uses connected with them.

In many viral infections, in particular in the HIV-1 infection, a strong immune defence is observed, which is in a position to control the virus over a period of several years. The period in which the virus is controlled and no illness symptoms are observed, is called the asymptomatic phase of the (HIV) illness. During the course of the illness, new virus variants repeatedly form. It is thereby possible for the virus to escape the human immune defence and to repeatedly infect new defence cells of the immune system (cf. M. Schreiber et al., J. Virol. 68 No. 6 (1994) 3908-3916; J. Gen. Virol. 77 (1996) 2403-2414; Clin. Exp. Immunol. 107 (1997) 15-20; J. Virol. 71 No. 12 (1997) 9198-9205).

In the prior art, for the treatment of viral illnesses, such as e.g. the polio virus (Horaud F et al., Biologicals, 1993, 21: 311-316), Hanta virus (Ulrich R et al., 1998 Vaccine 16: 272-280; Schmaljohn C S et al., 1992 vaccine 10: 10-13), Lassa virus (Morrison H G et al., 1989 Virology 171: 179-188; Clegg J C et al., 1987 Lancet 2: 186-188), hepatitis A virus (Clemens et al., 1995 J. Infect. Dis. 171: Suppl. 1: p. 44-p. 49; Andre et al., 1990 Prog. Med. Virol. 37: 72-95) and hepatitis B virus infection (McAleer et al., 1984 Nature 307: 178-180) but also the HIV infection (Egan et al., 1995 J. Infect. Dis. 171: 1623-1627, Kovac et al., 1993 J. Clin. Invest. 92: 919-928), only single, genetically unmodified, specific virus antigens or single inactivated virus strains have been used for studies of suitable vaccination strategies. In the case of HIV, for example, the external envelope proteins of two virus strains were up until now prepared for vaccine experiments on humans (MN and SF2) (Zolla-Pazner et al., J. Infect. Dis. 178 (1998) 1502-1506). Both GP120 molecules differ from each other in the amino acid sequence, but particularly in the amino acid sequence of the variable regions, such as e.g. of the V3 loop. Because of the different V3 loop sequences, the two virus strains have different phenotypic properties. The HIV-1 MN strain is a virus variant which preferentially infects macrophages and cells which possess the viral co-receptor CCR5. Viruses of the SF2 type, on the other hand, multiply preferentially in T cells and use the viral co-receptor CXCR4. Such viruses are therefore also called T cell trophs (e.g. HIV strain) or macrophagotrophs (e.g. HIV strain MN). Vaccination experiments have been carried out in chimpanzees with these two GP120 variants. It has been shown in these experiments that an immune response can be induced which not only protects against the two HIV strains MN and SF2, but is also capable of preventing infection by other virus strains, but also by HIV-1 patient isolates. In contrast to chimpanzees, only one of the two GP120 variants has been used in humans for vaccination experiments up until now. Neither MN GP120 nor SF2 GP120 offers certain protection against infection by a virus variant such as occurs in a person infected by HIV-1 (patient isolate or wild-type virus). J. A. Levy offers in “HIV and the Pathogenesis of AIDS”, Publisher: Jay. A. Levy, Chapter 15, 2^{nd} Edition, ASM Press Washington, D.C., 1998, an overview of the state of research in connection with vaccination with GP120 envelope protein. The disadvantage of the vaccination strategies discussed or examined up until now in the prior art is, among other things, that, with the vaccines used, one is not in a position to prevent the formation of ever-new virus variants during the course of the viral illness.

The object of the present invention is therefore to provide a vaccine which in particular is in a position to prevent or clearly reduce the formation of new virus variants during the course of a viral illness and to limit or prevent the possibilities that the virus can escape the human immune defence and repeatedly infect new defence cells of the immune system.

In order to achieve the object, the items described in the following claims are proposed according to the invention.

The subject of the present invention is thus inter alia a protein vaccine which comprises a mixture of viral protein molecules, the molecules being sequence variants of a single viral protein or of part of same.

### DESCRIPTION OF THE FIGURES

FIG. 1 V3 loop sequence data of HIV-1 patient isolates (PI) (wherein the first listed sequence is SEQ ID NO: 13 and subsequent sequences are SEQ ID NOs: 14-24).

FIG. 2a Amino acid sequences of the NL4-3 V2 loop (SEQ ID NO: 25) and NL4-3 V3 loop (SEQ ID NO: 26). The regions which are preferably to be varied are underlined.

FIG. 2b Variation of the V3 loop (SEQ ID NO:27). Data from the Los Alamos database.

FIG. 4 Schematic representation of the process for the preparation of the mixture of gp 120-expressing plasmid vectors.

1.) Preparation of the degenerated DNA fragments for e.g. the V3 loop

a.) Synthesis of single-stranded DNA

b.) Hybridization of two complementary oligonucleotides.

2.) Cloning of the V3 loop DNA fragments into pUC18 delta env

3.) Cloning of the env gene into the gp 120 expression vector pBSCenvATG

V3 loop fragments with degenerated sequence e.g.:

FIG. 5 Heterogeneity of the vaccine using the V3 loop as an example

V3 loop sequence:

Degeneration at protein level:

variants

Degenerated DNA sequence of the respective variable amino acid positions:

variants

Variability at protein level derived from the degenerated DNA sequence:

### DETAILED DESCRIPTION

According to the invention, sequence variants of a protein are understood to be those molecules which contain an amino acid sequence derived vis-a-vis a native viral protein or a part (fragment) of same, the variants differing from each other in that at least one amino acid can be exchanged at any chosen point of the sequence or parts of same. The sequence variants preferably contain several amino acid exchanges at various points of the sequence, which are responsible for the production but also the bonding of virus-neutralizing antibodies. The number and position of the exchanged amino acids depends on the amino acid variability of the regions of the gp120 which are observed during the cell culture of adapted and wild-type HIV isolates. According to the invention, the sequence variants have a heterogeneity at at least two amino acid positions of the sequence or parts of same. A heterogeneity at three to eight and preferably at more than eight amino acid positions is particularly preferred, all amino acids occurring at these positions being possible. The number of possible sequence variants which represent the vaccine in their totality result from the combination of the various amino acids which are possible per position.

The invention relates in particular to a vaccine which comprises a mixture of ≧10² sequence variants, i.e. a mixture of more than 10² molecules of various amino acid sequences (homologues). A vaccine is particularly preferred which comprises ≧10³ and preferably ≧10⁴ sequence variants.

Within the framework of the present invention it is assumed that the vaccine can comprise, along with the sequence variants, the protein per se from which the sequence variants are derived.

An anti-viral active ingredient is thus provided according to the invention which comprises virus-specific proteins which all differ in their sequences or parts of same. In order to achieve this, the protein-coding gene is newly synthesized in order to generate new cleavage sites for DNA-restriction enzymes, in order to permit the exchange of specific regions. By chemical synthesis of DNA fragments which code for the concerned protein section, a gene bank is then produced for the protein-coding sequence. The expression vectors which code for the protein, are then transfected as a mixture into cells. From these protein-producing cells the mixture of the various viral proteins can then be isolated which according to the invention represents the preferred active ingredient.

In the case of HIV as a viral illness, the chronic infection and the associated continuous damaging of the immune system during the course of the illness lead to the complete loss of the specific virus defence. Neutralizing antibodies which have the property of forming a specific, very strong bond with certain antigenic structures are part of the specific virus defence. Foreign antigens are thereby marked and their interaction with e.g. virus receptors are blocked. Such a specific virus defence are neutralizing antibodies against the V3 loop of the external GP120 envelope protein. Such anti-V3 loop antibodies are in a position to prevent the infection of cells. In animal models, it has been shown that an infection by HIV-1 can be prevented by administering a specific monoclonal V3 loop antibody. By administering the same monoclonal antibody, it was also possible to heal an existing infection.

In contrast to experimental systems, the development of ever-new virus variants is however observed in the natural course of HIV infection. Thus hundreds of different V3 loop variants are found in a single patient at one time, as the variation of the HIV-1 is particularly high precisely in the V3 loop. The V3 loop is an important dominant antigenic domain of the GP120. A very specific humoral immune response is therefore formed against each V3 loop. The result of the induction of a highly specific immune response against the V3 loop is that neutralizing antibodies against the V3 loop of the HIV-1 variant A are not in a position to neutralize the variant B and vice versa (Schreiber et al., J. Virol. 68 (1994) 3908-16, Abrahamsson et al., 4 (1990) 107-12).

In the asymptomatic phase of the HIV illness, the cell-free virus variants in the serum are neutralized by antibodies. Only later can virus variants be observed in the serum, which escape autologous neutralization. These V3 loop escape variants are no longer recognized by V3 loop antibodies and are thus no longer neutralized either. All other virus variants which are found in the serum of the patients are however recognized by autologous V3 loop antibodies. This indicates that during the course of the illness, virus variants occur against which no V3 loop antibodies exist. If the antibody content of serum samples of a patient is examined over a period of several years, the V3 loop antibodies against the V3 loop escape variant which occurs in the later stage can be detected in the serum samples which were taken at the beginning of the asymptomatic phase. The result is not, as with other infectious diseases, the standard escape of the pathogen through ever-new antigenic variation, but the switching-off of the neutralizing immune response to a virus replicating in the patient. The observed absence of the neutralizing V3 loop antibodies is thus the result of a continuous loss of the type-specific V3 loop antibodies. The corresponding virus can multiply through the loss of the neutralizing antibodies, which leads to an increase in the viral load in the serum of the patients. In the course of the illness, the increase in the viral load in the lymph nodes is also observed (Chun et al., Nature 387 (1997) 183-188).

A virus which establishes itself against the immune system in the patient and develops into the dominant virus variant must necessarily overcome all antiviral barriers which the immune system offers. Along with neutralizing antibodies, cytotoxic T cells are also in a position to suppress the virus reproduction. Cytotoxic T cells effect the death of HIV infected CD4+ T cells. Along with the loss of the neutralizing antibodies, the loss of such cytotoxic T cells against HIV infected cells is also observed (Zerhoui et al., Thymus 24 (1997) 203-219; Gould et al., Semin Cell Dev Biol 9 (1998) 321-328; Wagner et al., J Gen Virol 74 (1993) 1261-1269; O'Toole et al., AIDS Res Hum Retrovirsues 8 (1992) 1361-1368; Shearer et al., 137 (1986) 2514-2521). The object of a heterologous vaccine is therefore to induce or activate the mixture of many different GP120 envelope proteins of HIV both neutralizing antibodies and cytotoxic T cells against many different virus variants.

As already stated, the HIV illness is characterized in that the virus is constantly changing. This is effected by the error rate of the viral enzyme reverse transcriptase which commits errors during the increase of the viral genetic information. Mutants are thereby produced which are on the one hand selected because of their biological properties and the antiviral action of the human immune system. As with HIV, there are also other pathogens which transcribe their genetic information without error correction, which can lead to the formation of many variants. These include Hanta and Lassa viruses as well as hepatitis A, B and C viruses. Thus it is observed in people vaccinated for hepatitis B that vaccination fails in approx. 2%. In these 2%, hepatitis B “escape” variants are found which cannot be suppressed by the immune response induced by the vaccine.

The formation of vaccine, therapy and immune escape variants is based on the genetic variability of these viruses (Blum, Int J Clin Lab Res 27 (1997) 213-214; Jongerius et al., Transfusion 38 (1998) 56-59).

Up until now it has not been known in the state of the art how such a loss of virus-neutralizing antibodies can be countered. The strategies adopted to date were successful only in certain respects, i.e. in relation to the formation and the maintenance of individual variant-specific antibodies against specific viruses, but the treatment methods proposed to date in the form of antiviral medicaments or inoculants based on a memo-substance cannot counter the loss of type-specific antibodies against the variety of the protein variants formed by the virus in the course of the illness.

It is possible, through the present invention of a heterogeneous vaccine, to prevent the loss of neutralizing V3 antibodies or at least to clearly counter this loss.

On the basis of the present invention, for the first time an immunoreconstitutive treatment of virus-infected patients, in particular infected with HIV-1, is provided in which the immune system is activated such that the naturally acquired immune defence against the virus population of the various virus variants is regenerated and stimulated anew, in order to thus prevent the virus variants present in the patient from multiplying.

In short, the invention is based on the concept of preparing a mixture of GP120 proteins for an active ingredient against HIV-1, these GP120 proteins differing e.g. either in the V2 or the V3 loop or simultaneously in the two variable domains, i.e. the V2 loop and the V3 loop. In order to achieve this, the GP120-coding gene has to be newly synthesized accompanied by the formation of new (monovalent) cleavage sites for DNA-restriction enzymes which allow the specific exchange of the e.g. V2 and V3 region. By chemical synthesis of DNA fragments which code for the V2 loop and V3 loop a V2/V3 gene bank for GP120 is then produced. The GP120 expressions vectors are finally transfected into cells as a mixture, and the mixture of the various GP120 proteins which can be used as an active ingredient for the prophylaxis and/or therapy of the HIV illness or AIDS can then be isolated from these GP120-producing cells.

The consideration on which the present invention is based thus comprises the preparation for an immunotherapy or a vaccine against HIV-1 of many different GP120 molecules which carry V3 loop sequences (and/or V2 loop sequences) such as can also be identified in virus variants in patients.

The production of a mixture of natural virus variants in the cell culture system is very expensive. It must be borne in mind in particular that the composition of a virus mixture changes as cells which can be infected by HIV-1 have to be used in the cell culture. Some virus variants which have selective advantages, e.g. faster growth kinesis, therefore become dominant in the cell culture system after only a few days and expel the other slower virus variants. This applies above all when the various virus variants which are present in the peripheral mononuclear blood cells (PMBC) or in the serum of patients are to be isolated. As it is not possible to cultivate a constant mixture of HIV variants, neither can a corresponding mixture of GP120 proteins be produced nor isolated in this way from virus cultures.

Within the framework of the present invention, a genetic-engineering approach was therefore chosen for the preparation of HIV-1 variants and GP120 variants. For the preparation of virus variants and recombinant GP120, a cloning system was constructed which consists of two vectors. The central component of both vectors is a novel, chemically synthesized HIV-1 envelope protein gene (HIV-1 env gene) which codes for the GP120 protein. One vector contains the entire genome of HIV-1. After transfection of this vector into cells, the cells produce infectious virus particles. This facilitates the production of a mixture of virus variants as cells can be used which are resistant to an HIV-1 infection. Therefore, in such a cell culture system, the composition of the virus mixture cannot be changed, because of the biological properties of the virus variants. The second vector facilitates the expression of GP120-variants in eukaryotic cells. This vector serves directly for the preparation of the inoculant (the vaccine).

For the genetic-engineering preparation and expression of the V3 loop GP120 variants in eukaryotic cells, a special gene construct for HIV-1-env was created, called gene cassette in the following. For the preparation of the gene cassette, according to the invention the entire coding sequence of the env gene was chemically synthesized. With the help of this method it is possible to change the coding sequence of the gene as desired, new DNA-recognition sequences for DNA-cutting restriction enzymes being able to be incorporated into the env-gene sequence. It must be borne in mind that the amino acid sequence of the original GP120 (preferably of the strains NL4-3 and PI-932) does not change, while new restriction cleavage sites form in the DNA sequence of the env gene. At the same time, according to the invention cleavage sites for enzymes which repeatedly occur in the env sequence are removed so that in each case there is only one cleavage site for a specific restriction enzyme, such as e.g. BglII. According to a preferred version of the invention ten new, once-occurring (monovalent) cleavage sites are inserted at intervals of approx. 150 base pairs. The new env gene produced in this way is also called gene cassette within the framework of the present invention. In principle, the new env fragment is similar to a polylinker.

The cleavage sites for the restriction enzymes BstEII and BamHI define the gene cassette preferred according to the invention (SEQ ID NO: 9). In order to be able to exchange the region of the env gene, both cleavage sites are present only once in the pBSCenvATG expression vector (SEQ ID NO: 10) for gp120 and in retroviral vector pNL4-3. The vector, given the reference code pBSCenv-V3 by the depositor, was deposited on Jan. 6^{th} 1999 at the DSMZ-Deutsche Sammlung von Mikroorganismen und Zellkulturen GmbH, Mascheroder Weg 1b, 38124 Brunswick, Germany, under the accession number DSM 12612 in accordance with the Budapest Treaty.

Through the exchange of the gene section defined by BstEII and BamHI, the newly created sections of the env gene which are likewise defined by BstEII and BamHI, can be cloned into both vectors. The PstI/Bc1I V2 loop and the Bg1II/XbaI V3 loop env fragment are chemically or enzymatically synthesized and then cloned into a standard vector such as e.g. pUC 18 or 19. In this vector, the fragment is checked by sequencing and then cut out of the vector by a Pst/BcII or BgIII/XbaI digestion and transferred into the pBSCenvATG and then into the pNL4-3 vector. In place of the intermediate cloning into a standard vector (pUC 18, pUC 19 etc.), the PstI/Bc1I V2 loop and BglII/XbaI V3 loop fragments can also be cloned directly into pBSCenvATG.

As there is a cleavage site for a restriction enzyme every ca. 100 base pairs in the gene cassette, all regions of the env gene, in particular the V3 loop or V2 loop, can be exchanged for any DNA fragments. The section coding for the V3 loop can be exchanged for a Bg1II-XbaI fragment, which has a size of 244 base pairs (cf. SEQ ID NO: 9; nucleotides 708 to 955). The aim is to exchange the gene sections which code for the variable loops of the GP120 protein for new DNA fragments. The fragments are synthetically produced and then cloned into the env gene cassette. If the sequence is varied at certain positions during the synthesis of the V2 loop and V3 loop DNA fragments, i.e. all four nucleotides are incorporated equally or the nucleotide inosine is used at this position, env-gene variants with a pre-set variation of the thereby-coded amino acid sequence can be produced. The V3 loop sequences of patient isolates are the starting point for the preparation of the GP120 mixture. If these variants are cloned into the gp120 gene cassette, the corresponding GP120 proteins can be expressed with the variable antigenic domains in eukaryotic cells. Sequence data of HIV-1 patient isolates are available for the variation of the V3 loop region of the env gene. Patient isolates are virus variants which have been cultivated direct from patient material, serum or infected cells from the blood in the laboratory. These viruses are characterized, unlike HIV-1 viruses which are cultivated in the laboratory for a long time, by particular properties. These viruses are more resistant to neutralizing antibodies and chemokines. In contrast to cell culture-adapted viruses, patient isolates use different co-receptors for the infection of cells. As patient isolates differ greatly from laboratory-adapted viruses, the collected V2 loop or V3 loop sequence data of the env genes of such viruses are to be used for the preparation of the GP120 mixture. Within the framework of the invention, additional base exchanges are possible in regions outside the sequence sections coding for V2 and V3.

According to a particular version of the invention, a protein vaccine is made available which comprises a mixture of GP120 proteins of the human immunodeficiency virus (HIV) which in each case differ from each another in their amino acid sequence in the region of the V3 loop and/or of the V2 loop.

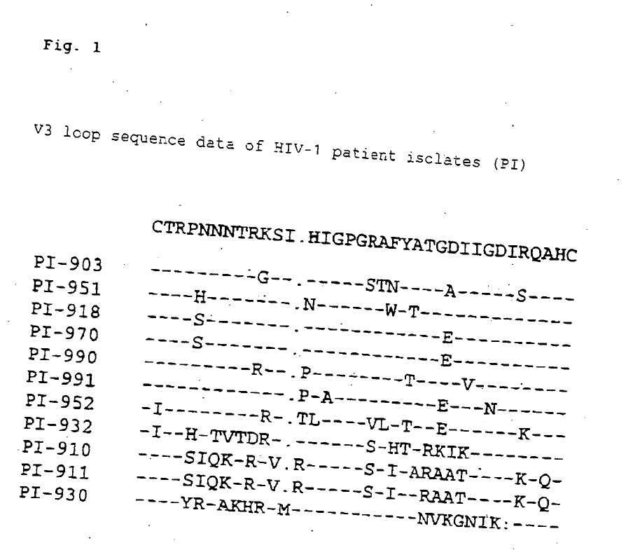

Within the framework of the present invention, during the genetic-engineering preparation of the protein mixture or the protein vaccine, sequence variations are introduced preferably in regions of the V3 loops which lie outside consensus sequences (cf. e.g. M. Schreiber et al., J. Virol. 71 no. 12 (1997) 9198-9205). The consensus sequences are sequence sections which essentially remain both between different virus strains and during the formation of virus variants as a viral illness progress (HIV illness) in the body. Such a section is the sequence Gly-Pro-Gly-Arg-Ala-Phe (GPGRAF) in the case of the HIV-1 of B sub-type. To the left and right of this sequence unit lie short regions 4-10 amino acids long which can vary greatly. In FIG. 1 this is represented using as example the V3 loop sequence variations of patient isolates.

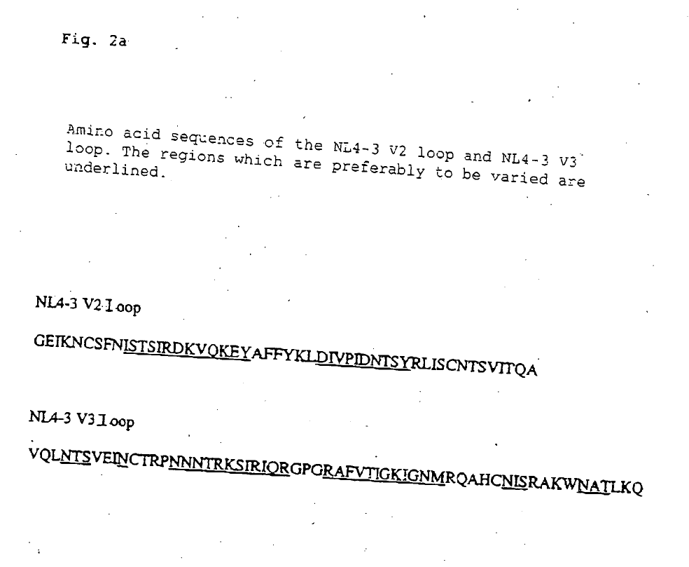

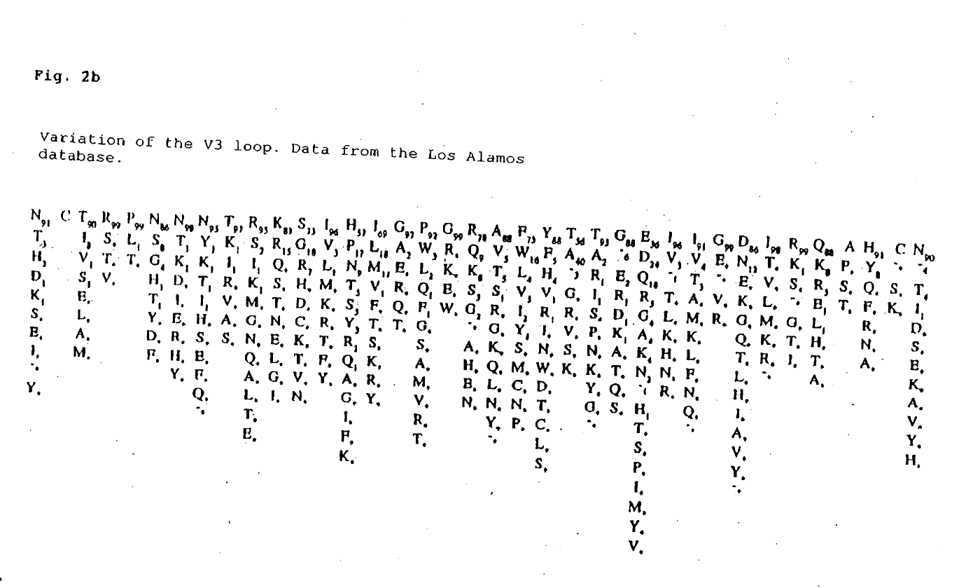

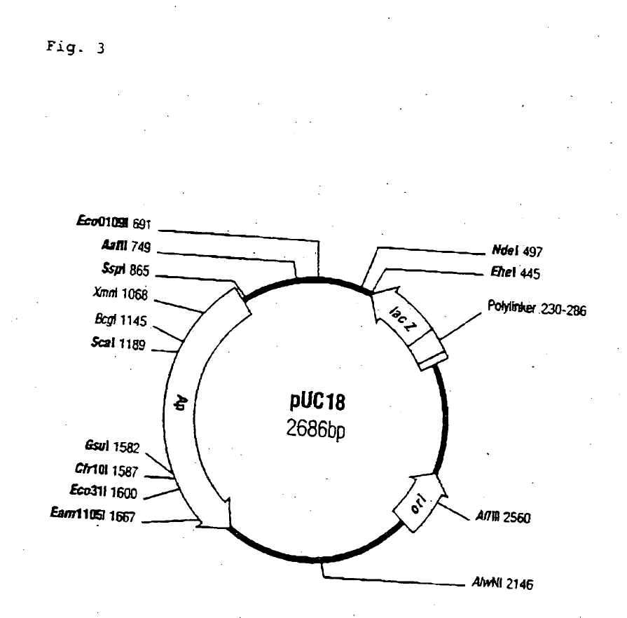

In addition to variations in the region of the V3 loop, the molecules of the protein vaccine according to the invention can also contain sequence variations in the region of the V2 loop. Changes in the amino acid sequence outside consensus sequence regions are preferred in this case also (see FIG. 2). Furthermore, additional amino acid exchanges are possible in regions outside the V2 and V3 loops.

In connection with the present invention the normal one- or three-character codes are always used to describe the amino acids. When there is variation of the amino acid sequences inside the protein vaccine according to the invention, any desired amino acid exchanges are possible. In each case, however, amino acids of the viral GP120 sequence or of the sequences of the V2 loop and V3 loop are preferably exchanged for amino acids which also occur in other virus variants at the corresponding sequence positions (see FIG. 2, position of 1997 sequence data in: Human Retroviruses and AIDS, A compilation and analysis of nucleic acid and amino acid sequences, Los Alamos National Laboratory, Los Alamos, N.M. 87545, U.S.A., Editors: B. Korber, B. Hahn, B. Foley, J. W. Mellors, T. Leitner, G. Myers, F. McCutchan, C. Kuiken).

Within the framework of the present invention, for the first time a protein vaccine is made available which is prepared by genetic engineering and comprises sequence variants of specific viral antigens (proteins). The preparation of the vaccine is described in the following using the V3 loop as an example. It is self-evident to the person skilled in the art that the synthesis principle described below can be transferred to other viruses or viral proteins or parts of their sequences.

In short, a mixture of GP120 proteins with variable V3 sequences which are modelled on the variability of the V3 loop of the GP120 protein of HIV is prepared as a basis for the vaccine.

For this, the sequence coding for the GP120 protein is firstly cloned piecewise into the plasmid pUC 18/19. This takes place through a polylinker exchange so that all interesting sections with monovalent restriction sites are flanked by the possible restriction sites which were previously inserted in the gp120 sequence by silent mutations, and thus can be subsequently cut out and exchanged.

Two homologous nucleic acid oligomers are then prepared with the help of a DNA synthesizer (length approx. 300 base pairs). After hybridization has taken place, the double-stranded DNA fragment forms with the DNA sequence of the V3 loop. Various standard methods from molecular biology can be used for the preparation of the double-stranded V3 loop DNA fragment. In one method, the variables are introduced by inosines and in the case of the corresponding complementary strand the variables are added by a nucleotide mix (AGCT, AGC, AG, . . . ). In this method, the DNA fragment is prepared by exclusively chemical means. In another method, a combination of chemical and enzymatic DNA synthesis, the variables are introduced by nucleotide mixtures. The oligonucleotides are then hybridized at complementary ends of a length of 20-30 bases, which are not variable. The synthesis to produce the completely double-stranded molecule takes place enzymatically using a DNA polymerase. Both isothermal DNA polymerases (Klenow fragment) or thermostable DNA polymerases (Taq-polymerase)can be used. If Taq polymerase is used, then larger amounts for the cloning of the V3 loop DNA fragment can also be prepared using the polymerase chain reaction. Using these methods, a double-stranded DNA fragment is prepared which is variable at specific positions. This degenerated DNA sequence codes for the corresponding variety of V3 loop amino acid sequences of the GP120 protein mixture to be prepared, the protein vaccine.

The mixture of the synthesized V2 fragments has a PstI cleavage site at the 5′ end and a Bc1I cleavage site at the 3′ end. The mixture of the synthesized V3 fragments has a BglII cleavage site at the 5′ end and an XbaI cleavage site at the 3′ end. Using the two respective cleavage sites, the fragment mixture is cloned into a vector, e.g. pUC18 delta-env or pUC 18 BstEII-BamHI (FIG. 3). After this cloning a mixture or a pool of plasmid DNAs of the vector pUC 18 forms, which all have the complete BstEII-BamHI env fragment. All plasmid DNAs differ exclusively in the sequence of the env V2 loop or V3 loop. By inserting the V2 or the V3 fragments, the deletion (delta env) of the env gene section in the vector pUC18 is stopped.

This thus-prepared mixture of the plasmid DNA with variable env fragments is transformed into E. coli and fermented. E. coli amplifies and replicates this mixture of plasmid DNA. All possible variables of these V2/V3 loop plasmids are produced.

This plasmid pool is then isolated. The env fragment is then cut out of the mixture of the plasmid DNA by BstEII and BamHI digestion and cloned direct into the vector for the expression of the viral gp120. This vector has the advantage that the GP120 is also expressed in the eukaryotic cell system.

This pool of BSCenvATG-vector DNA with variable gp120 construct is now transfected into Cos or Chinese hamster ovary cells (CHO cells). These eukaryotes then express this pool of plasmids so that statistically the corresponding protein is translated to each variable and then—depending on which eukaryotes are used—correspondingly glycosylated. The proteins are then harvested, followed by purification to give the finished product (protein mixture or GP120 mixture with variable amino acid sequence).

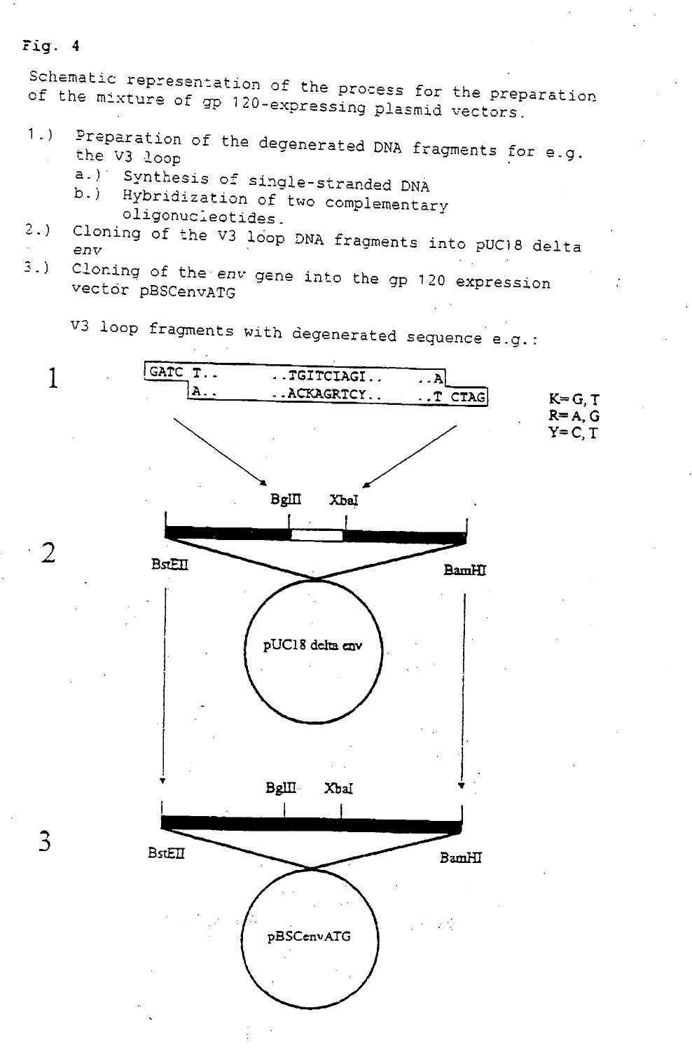

By way of illustration, the process for producing the protein vaccine according to the invention is represented clearly in FIG. 4.

As already shown, variations in the V2 loop and/or in the V3 loop can be carried out. The variation of the V2 loop can take place according to the same scheme as described for V3.

The preparation of the protein vaccine according to the invention takes place, as described above, via various key constructs, i.e. nucleic acid intermediate steps and DNA constructs, which are essential for carrying out the invention.

As already explained, the nucleic acid sequence coding for the GP120 protein is cloned piecewise into pUC 18/19, the starting point according to the invention being a gp120 sequence in which the repeatedly present restriction sites are firstly modified by silent mutations such that each of the remaining restriction sites still occurs only once in the sequence. This sequence modification is necessary in order to be able to insert the expression cassette, which is necessary for producing the sequence variants, selectively at a quite specific point which is defined by two restriction sites each occurring only once in the sequence. Methods for the introduction of silent mutations in a nucleic acid sequence are well known to a person skilled in the art.

A subject of the present invention is therefore also a nucleic acid sequence which is derived from the env sequence in SEQ ID NO: 1 or a fragment of same, it being modified such that it contains exclusively monovalent restriction sites. Preferably, the modification takes place by the introduction of silent mutations. According to a preferred version of the invention, the nucleic acid sequence has the sequence shown in SEQ ID NO: 9. The sequence of the gene cassette can be modified such that it contains the total env gene or parts of the env gene of virus isolates from patients. Preferably, such a gene cassette is to contain the env sequence of the patient isolate PI-932 (SEQ ID NO: 11). A gene cassette preferred according to the invention based on the PI-932 sequence is shown in SEQ ID NO: 12.

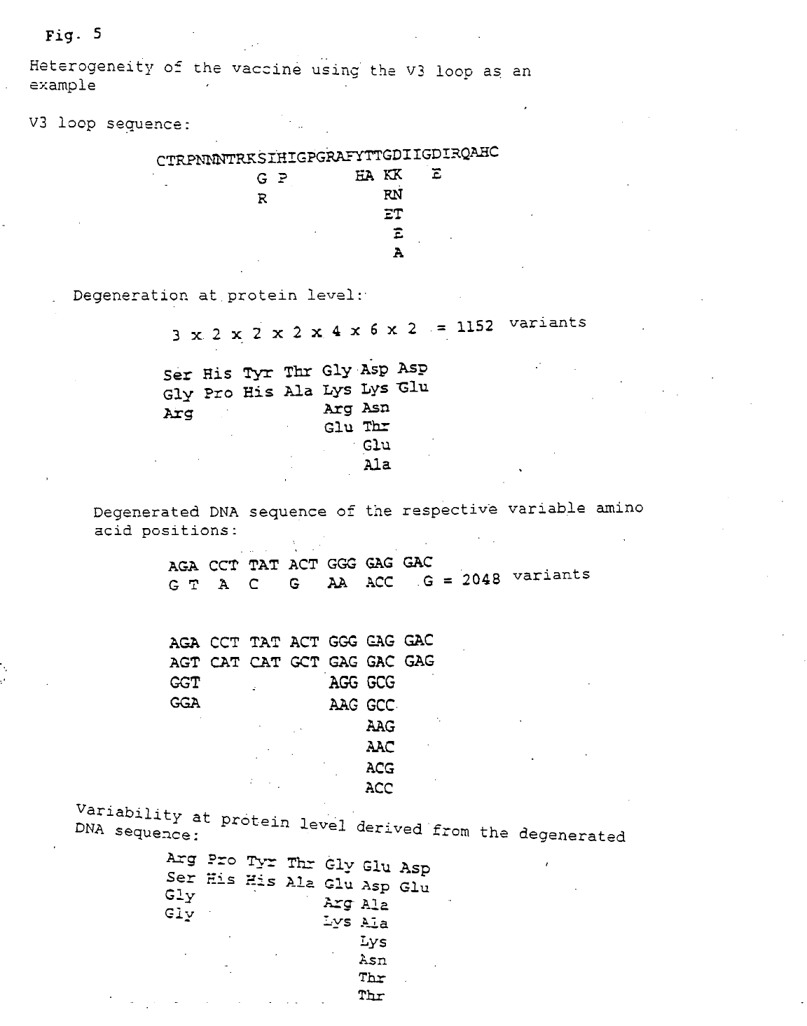

A subject of the present invention is therefore also a single-stranded nucleic acid sequence which contains the region coding for the V3 loop and/or for the V2 loop or fragments or parts of same in which, in the case of the V3 loop, a Bg1II-XbaI (247 bp) or also a Bg1II-NheI (283 bp) fragment can be exchanged for a modified fragment which carries nucleic acid exchanges or mutations at least 6, preferably at 9 to 20 positions, and in the case of the V2 loop, a PstI-Bc1I (139 bp) or also a PstI-EcoRI (339 bp) fragment for a modified fragment which carries nucleic acid exchanges or mutations at least 6, preferably at 9 to 20 positions. The nucleotides within a nucleic acid sequence are in each case replaced either by inosine or by a mixture of 2-4 nucleotides. This is explained using the example in FIG. 5. If various amino acids are to be combined at 7 amino acid positions of the V3 loop 21 to give 1152 variants, two nucleotides must be introduced by chemical synthesis at each of 11 nucleic acid positions into the sequence of the single-stranded nucleic acids (oligonucleotides) (FIG. 5).

The single-stranded nucleic acid sequences are—as already mentioned above—hybridized or synthesized to give the double strand.

The present invention therefore furthermore relates to double-stranded DNA which comprises hybrids of the above-mentioned single-stranded nucleic acid sequences, where in each case one nucleic acid sequence (5′-3′ oligomer or 3′-5′ oligomer) contains inosines at one or more selected positions, and the other nucleic acid sequence (3′-5′ oligomer or 5′-3′ oligomer) in each case contains two, three or four of the possible nucleotides (adenine, A; thymine, T; guanine, G; cytosine, C) at the corresponding complementary positions during the later hybridization. Sequences are thereby produced in which the four bases are present randomly distributed at the respective positions of the single-stranded nucleic acid sequence (DNA) so that the desired combinations of A, T, G or C are produced for the corresponding desired amino acid codons (a codon is formed from a series of three nucleotides). Because of the variation of nucleotide positions, randomly distributed sequence combinations result. Therefore, e.g. for the combination of (A, C) (ACT) (ACGT) a total of 2×3×4=24 possible DNA sequences form, which code for various amino acids. At the same time, the number of variable positions also determines the number and position of the introduced inosines in the complementary DNA sequence. The calculation of the heterogeneity of such a construct is shown in FIG. 5.

As, to form the double-stranded DNA, singled-stranded DNA containing inosines is in each case hybridized with oligomers containing A, T, G or C in each case randomly distributed at the corresponding positions, a mixture of double-stranded gp120-sequence or of part (fragment) thereof is obtained, the nucleic acid sequences in each case being derived from the env sequence (SEQ ID NO: 1 or 12) and the nucleic acid sequences in each case being distinguished from each other in the region coding for the V2 loop and/or in the region coding for the V3 loop. This means that nucleic acid sequences contained in the mixture differ from each other such that they code for a mixture of proteins which in each case contain amino acid sequences which are different from each other in the V2 loop and/or in the V3 loop.

According to a preferred version of the invention, in this way, at least 10², preferably at least 10³ and according to a particularly preferred version of the invention at least 10⁴ sequence variants can be obtained at nucleic acid level (DNA level). This DNA mixture is hereafter called the DNA pool, and in relation to the variants of the gp120 sequence this is hereafter called the pool with variable gp120 construct (gp120 pool).

Within the framework of the present invention, the term sequence variants of a DNA sequence is taken to mean molecules which have a derived nucleic acid sequence vis-a-vis a native viral DNA molecule or a part (fragment) of same, the variants being distinguished from each other in that at least one nucleotide can be exchanged at any chosen points of the sequence. Preferably, the sequence variants contain several nucleotide exchanges at various points of the sequence, the number and the position of the exchanged nucleic acids essentially depending on the length of the nucleic acid sequence.

Upon the expression of the env-gene variants starting from the produced plasmid-DNA mixture, it is to be expected that, because of the random distribution, all possible DNA sequences are expressed. Therefore, all sequence variants that are possible, on the basis of the pre-set DNA mixture, of the gp120 molecule or of part of same are then also formed. The heterogeneity of the gp120 mixture is calculated on the one hand using the heterogeneity of the DNA sequence and the degeneration of the genetic code for the amino acids (cf. also FIG. 5).

The heterogeneity of the plasmid-DNA mixture is demonstrated by DNA sequencing of single clones. To this end, the mixture of the plasmid DNA is transformed in E. coli and single clones are randomly selected and their V2 or V3 loop sequence determined. In total, approx. 100-200 different clones are to be sequenced. The statistical distribution of the DNA sequences can be use to calculate the distribution of the whole mixture. The method corresponds very largely to the method of random sampling such as is used as standard for the quality control of the most varied products.

The direct molecular analysis of the heterogeneity of the GP120 protein mixture proves to be somewhat more difficult, as single GP120 molecules cannot be separated from the mixture and identified. This is in the nature of things, as the individual GP120 variants differ from each other in a few amino acids only. Gel electrophoresis separation or mass spectroscopic analysis can establish whether a mixture or a single form of the GP120 molecule is involved. When the GP120 mixture is used e.g. in animals as a vaccine, the immune response induced in the animal is directly dependent on the number and the composition of the GP120 mixture. The immune response of the animal, the neutralizing antibodies can then be examined in virus neutralization tests. To monitor a corresponding variant-overlapping immune response, various patient isolates of HIV-1 can be used in the neutralization test. Preferably, patient isolates are chosen which differ in the ability to use various co-receptors for the infection of the target cells. The neutralization potential of the GP120 mixture then serves as a quality measure.

A subject of the present invention is therefore furthermore a protein mixture which comprises GP120 proteins which contain amino acid sequences which in each case differ from each other in the V2 loop and/or in the V3 loop, the mixture containing, according to one version of the invention, at least 10^{2,} preferably at least 10³, according to a version of the invention, and, according to a particularly preferred version of the invention, at least 10⁴ sequence variants.

As already mentioned earlier, the mixture of double-stranded DNA which can be obtained by hybridization of inosines-containing single-stranded DNA with randomly distributed A-, T-, G- and/or C-containing single-stranded DNA, i.e. the pool with variable GP120 construct, is transformed and fermented in prokaryotic or eukaryotic host cells, preferably E. Coli.

A subject of the present invention is therefore plasmids which contain inserted double-stranded DNA, in each case comprising hybrids of the single-stranded, inosine-containing nucleic acid sequence (see above) with the single-stranded nucleic acid sequence containing a mixture of all four nucleotide variants (A, T, G, C) (see above). Furthermore, the invention relates to a vector mixture which contains a mixture of these plasmids, the nucleic acid sequences of the plasmids in each case differing from each other in the region coding for the V3 loop and/or in the region coding for the V2 loop. According to one version of the invention, the vector mixture comprises at least 10², preferably at least 10³ and according to a particularly preferred version of the invention at least 10⁴ of the named plasmids. Depending on which host cells are to be used to express the vectors (plasmids), various expression systems and base vectors well-known to a person skilled in the art can be considered (cf. Methods in Enzymology, Vol. 185, Gene Expression Technology, 1991, Publ. D. V. Goeddel, Academic Press, Inc.). Thus, for the expression in E. coli, the plasmid pUC 18/19 is preferred as base plasmid into which the nucleic acid sequence variants are cloned. Within the framework of the present invention, the vector mixtures can thus be expressed either in bacterial host cells such as E. coli, or in eukaryotic host cells, preferably from the group consisting of Cos, CHO or baby hamster kidney cells (BHK cells) or in other host cells.

The present invention thus furthermore relates to E. Coli host cells or eukaryotic host cells which are transfected with a vector mixture according to the invention.

As host cells are transfected with the DNA pool (the vector mixture), it is to be expected that as great or at least approximately as great a number of differently transformed host cells forms as there are sequence variants present in the DNA pool. Upon the preparation of the vaccine, special attention is to be paid to the yields during the individual cloning steps. The yields upon preparation of the double-stranded DNA fragments, the ligation of the V3 loop and V2 loop DNA fragments into the env gene and the transformation or the preparation of bacteria and cell clones must be such that the heterogeneity that is to be achieved of the mixture is not restricted. This is to be illustrated using an example. If, e.g. a number of balls are to be removed from a quantity of balls which differ on the basis of two colours, but there is to be a 99.9% probability that both colours are present in this selection, approx. 13 balls would have to be removed (G. Schreiber, Ein kombinatorisches Problem aus der Genetik; Bio-engineering 1988, 2: 32-35). Transferred to the cloning steps of the HIV vaccine, this means: If a heterogeneity of approx. 6000 virus variants is to be produced, 13 times as many clones would have to be produced in each individual cloning step. Thus, a gene bank of approx. 80,000 clones must be prepared for the V3 loop. Starting from this gene bank, the expression vectors for the transfection of the CHO cells are then prepared. During transfection of the CHO cells, consequently, approx. 80,000 transfections must then be achieved. Such a mixture of transfected CHO cells then produces the desired quantity of 6000 different gp120 variants.

The present invention also relates to a process for the preparation of a nucleic acid sequence coding for a viral protein (expression cassette) in which the sequence is so modified, or preferably so many silent mutations are introduced, that it then contains at least two and preferably exclusively monovalent restriction sites. Particularly preferably, the sequence still contains only monovalent restriction sites. Preferably, the protein coded by the nucleic acid sequence is GP120, the env wild-type sequence coding for the GP120 being varied by silent mutations.

A further subject of the present invention is a process for the preparation of the vector mixture according to the invention, which preferably contains a mixture of plasmids, the nucleic acid sequences of which differ from each other in the region coding for the V2 loop and/or in the region coding for the V3 loop, the plasmids according to the invention being ligated into a (base) vector which can be expressed in host cells (preferably E. coli, Cos, CHO or BHK cells). The (base) vector is preferably the pUC 18, pUC 19 or BSCenvATG vector.

Within the framework of the present invention, a process is furthermore made available for the preparation/production of host cells, preferably chosen from the group consisting of E. coli, Cos, BHK or CHO cells, in which the host cells are transformed with a vector mixture according to the invention, which contains a mixture of plasmids the nucleic acid sequences of which in each case differ from each other in the region coding for the V2 loop and/or in the region coding for the V3 loop.

Finally, according to the invention, a process is for the first time made available for the preparation of a DNA vaccine, in which the process according to the invention for the preparation of the vector mixture is carried out, the plasmids according to the invention expressing the mixture of the gp120 proteins after application in host cells (e.g. human and animal monocytes). The DNA vaccine can optionally be formulated with pharmaceutically compatible auxiliaries and/or carriers for application and after administration in the organism, make possible the production of the sequence variants of viral proteins.

The present invention thus also relates to a pharmaceutical composition or a DNA vaccine which codes for a mixture of structurally different virus proteins, the vaccine containing a mixture of sequence variants of a viral DNA molecule or of part of same, i.e. of DNA molecules the nucleic acid sequences of which differ from each other in the region coding for the protein, a part or a fragment of same. By the term “structurally different virus proteins” is meant according to the invention those proteins the amino acid sequences of which are derived from the wild-type sequence of the corresponding virus protein, the amino acid sequences deviating from each other insofar as they differ from each other and compared with the wild-type sequence through one or more exchanged amino acids at identical or different positions of the sequence. As already mentioned, the nucleic acid sequences in the vaccine code preferable according to the invention for a mixture of structurally different GP120 proteins of HIV (sequence variants of GP120), a vaccine being particularly preferred which contains a mixture of DNA molecules, the nucleic acid sequences of which differ from each other in the region coding for the V2 loop and/or in the region coding for the V3 loop of gp120 of the HIV-1. In this context, reference is also made to FIG. 3 in which known structural differences or variations in the V3 loop of GP120 are shown.

The DNA vaccine according to the invention is of particular importance within the framework of a gene therapy.

Finally, according to the invention, a process for the preparation of a pharmaceutical composition or a protein vaccine is for the first time made available in which the host cells according to the invention, i.e. host cells which are transformed with a vector mixture according to the invention, the vectors in each case containing plasmids, the nucleic acid sequences of which differ from each other in the region coding for the V2 loop and/or in the region coding for the V3 loop, are cultivated under conditions which allow the expression of the mixture of viral protein sequence variants. The host cells are preferably bacterial host cells, such as E. coli, or eukaryotic host cells, preferably from the group consisting of Cos, Chinese hamster ovary (CHO) or baby hamster kidney cells (BHK cells).

Through the present invention, it is thus possible to make available a vaccine comprising variable GP120 proteins which, in the prophylactic and/or therapeutic treatment of a HIV infection or AIDS
1. activates the immune system,
2. induces the formation of HIV-l-neutralizing antibodies,
3. prevents the loss of neutralizing antibodies and
4. in which monitoring of the stimulation of the GP120-specific immune response is taken over by the active ingredient.

This is preferably carried out in that, even before the loss of HIV-neutralizing antibodies associated with the outbreak of AIDS, sufficient antigens are available which, because of their variety of different amino acid sequences (i.e. their sequence variations) are in a position to induce the formation of those HIV antibodies which normally, i.e. without corresponding prophylaxis according to the present invention, are lost or experience a reduction in concentration as the illness progress. In addition to a preventative treatment, the present invention also relates to therapeutic treatment, the GP120 mixture (protein vaccine) being used, to counter a reduction or a loss of HIV-neutralizing antibodies by activation of the immune system and induction of the formation of new or additional HIV-1-neutralizing antibodies.

According to the invention, the mixture of structurally different viral proteins which are sequence variants of a viral protein (preferably of GP120) or of part of same is therefore used for the preparation of a vaccine for the prevention and or therapy of a viral infection in humans. Furthermore, the present invention relates to the use of a mixture of DNA molecules which code for sequence variants of a viral protein or of part of same for the preparation of a vaccine for the prevention and/or therapy of a viral infection in humans. According to a preferred version, the invention relates to the use for the preparation of a vaccine for the prevention and/or therapy of a HIV infection in humans.

Within the framework of the present invention, the virus infection can be any infection in which self-replicating virus variants form in the course of the illness, where those amino acid sequence sections or nucleic acid sections coding for the latter can be considered according to the invention as selected protein sequences or protein-coding nucleic acid sequences, in which, in the course of viral illnesses, variants, i.e. sequence variations, are always or often observed. Preferably, in the present case, these are sequences variants of the GP120 molecule (at protein level) or of the region coding for GP120 (at DNA level) or parts of same, in particular sequence variants in the V3 loop or in the V2 loop, preferably both in the V3 and in the V2 loop.

According to the invention, pharmaceutical compositions or vaccines are consequently made available which, in the immunoreconstitutive treatment of those suffering viral infection, activate the immune system in such a manner that the naturally acquired immune defence is regenerated against the virus and restimulated to thus prevent the virus variants replicating in the patient from multiplying. According to a preferred version of the invention, vaccines for the immunoreconsitutive treatment of those infected by HIV-1 are made available for the first time.

Within the framework of the present invention, the vaccines can either be formulated or administered per se, i.e. as DNA and/or protein mixtures without further additives, or together with further active ingredients, such as e.g. immunostimulants such as interleukin-2, CC and CXC-Chemokine, and/or pharmaceutically compatible auxiliaries and carriers. As a rule, the vaccines according to the invention are formulated for intravenous application, such as e.g. by the intravenous, intramuscular, subcutaneous routes. In addition, the vaccines can be formulated for oral, mucosal (intravaginal, intrarectal) and transdermal application.

The present invention has the following advantages:

Through a selective genetic manipulation of the nucleic acid sequence, with the help of a gene cassette, any desired variants of a pathogen or of a gene of the exciter can be prepared. The variations preferably affect the regions against which the neutralizing antibodies or cytotoxic T cells are formed. An inoculant in the form of a mixture of variants of the pathogen or of variants of the corresponding antigens of a pathogen can thereby be prepared. The advantages of this concept in relation to HIV as a pathogen lie in the preparation of a mixture of HIV-GP120, the external membrane protein of HIV, against which the virus-neutralizing immune response of humans is directed. As, in the precise case of the HIV illness, each patient is exposed to a variety of HIV variants which all differ in the sequence of the GP120 protein, the use of a variety of GP120 variants as inoculant is of particular advantage, this GP120 mixture being able to be used both for the immunization of normally healthy persons and for the therapy of persons already infected by HIV. In the case of immunization with the GP120 mixture, as broad as possible an immune response against as many virus variants as possible in humans is induced, which in the ideal case protects against all HIV variants. In the case of therapy with the GP120 mixture, the loss of neutralizing antibodies and the loss of the HIV-specific cellular immune response can be combated.

According to the invention, it is furthermore possible for the first time to prepare gp120 clones which, in the nature or variety of the observed sequence variations, correspond to plasma isolates of those infected with HIV or ill with AIDS (cf. M. Schreiber et al., J. Virol. 68 No. 6 (1994) 3908-3916). In this context, in contrast to various approaches followed to date, no isolated GP120 molecule or an antigenic fragment of same is used for immunization, but a protein mixture which consists of a variety of sequence variants which are characterized by a complete GP120 sequence which in addition contain the correct tertiary structure corresponding to the natural plication of the GP120 molecule. The conformation of the virus variants made available in the vaccine is important insofar as sequence and structure variants are thereby provided which, with the GP120 variants detectable in the course of the viral illness, as great as possible a similarity with regard to the observed sequence variations and also the conformation required for the bonding to CD4 and the co-receptor is achieved, whereby an effective immune stimulation can be achieved.

Through the invention, a mixture of GP120 proteins is provided which represents the protein vaccine. At the same time, a mixture of the genes coding for this protein mixture is provided through the invention. These genes can be transferred into a vector which is suitable for direct application in humans (DNA vaccine) (Ulmer et al., 1995 Ann NY Acad Sci 772: 117-125; Donnelly et al., 1995 Ann NY Acad Sci 1995 772: 40-46). A DNA vaccine has the advantage that the heterogeneity of the mixture of the DNA vectors can be greater than that of the mixture of the recombinant GP120 proteins. It is easier to produce a high heterogeneity of a DNA vector mixture. Such a DNA vaccine would cover a much broader spectrum of HIV variants. From the technical point of view, the preparation of the DNA mixture is easier. A precondition for the DNA vaccine is a gp120 expression vector which carries the two cleavage sites BstEII and BamHI. This makes possible the conversion of the V3 loop and V2 loop variables env gene fragments into such a vector.

With regard to the GP120 proteins, it is to be borne in mind that 5 potential points for N-glycosylation are located in the region of the V3 loop. The sugar groups protect the V3 loop against recognition by neutralizing antibodies. Virus variants which contain a glycosylated V3 loop are more poorly neutralizable than virus variants in which glycosylation is incomplete. As virus variants with a complete V3 loop glycosylation can escape the neutralizing immune response, they are important for the transfer and for the establishment of the infection. In the course of the illness, if a part of the neutralizing antibody response is lost, virus variants whose V3 loop is not completely glycosylated establish themselves. As the V3 loop is no longer covered by sugar groups, these virus variants replicate faster than the completely glycosylated V3 loop mutants. According to the invention, it is therefore advantageous to take the varying glycosylation of the GP120 V3 loop into account when constructing the GP120 vaccine.

Within the framework of the present invention, the principle represented with reference to HIV can be transferred to other viruses which also form variants in the course of the viral illness. In particular, vaccines can be provided against a variety of viruses which induce a broad immune response against many virus variants in humans which in the ideal case protects against all variants. In the case of therapy with such vaccines, the loss of neutralizing antibodies and the loss of the HIV-specific cellular immune response can be effectively combated. In this context, it is furthermore possible or even advisable, with pathogen types (viruses) which form several sequence variants of several proteins in the course of an illness, to provide several gene cassettes according to the invention each of which contains the nucleic acid sequence coding for variants of each of these proteins or fragments of same, in order to combat the loss of neutralizing antibodies against all conceivable virus variants.

Within the framework of the present invention, the advantages of a protein vaccine and a DNA vaccine can be advantageously combined in order to increase the success of a preventative and/or therapeutic treatment of a viral illness. A pharmaceutical composition is thus furthermore provided for the prevention and/or therapy of a viral infection, which comprises a protein mixture and a nucleic acid mixture, the protein mixture comprising sequence variants of a viral protein or of part of same, and the nucleic acid mixture comprises DNA molecules which code for sequence variants of a viral protein or of part of same. In particular, the pharmaceutical composition is a combination preparation which comprises both a protein mixture comprising one of the above-named sequence variants of the GP120 protein and a nucleic acid mixture derived from an above-named env sequence in SEQ ID NO: 1 or SEQ ID NO: 11 or a fragment of same.

The present invention is also described, without limitation, in the following numbered paragraphs:

(1.) Protein vaccine which comprises a mixture of viral protein molecules, characterized in that the molecules are sequence variants of a single viral protein or of part of same, the mixture containing≧10² sequence variants, which is obtainable by expression of a plasmid-DNA mixture which, because of the variation of nucleotide positions, contains randomly distributed sequence combinations.

(2.) Protein vaccine according to paragraph (1.), characterized in that the mixture contains≧10³ and preferably≧10⁴ sequence variants.

(3.) Protein vaccine according to paragraph (1.) characterized in that it comprises a mixture of GP120 proteins of HIV which in each case differ from each other in their amino acid sequence in the region of the V2 loop and/or of the V3 loop.

(4.) DNA vaccine which codes for a mixture of structurally different virus proteins, characterized in that the vaccine contains a mixture of sequence variants of a viral DNA molecule or of part of same, the mixture containing >10² DNA molecules which differ from each other in their nucleic acid sequence, where the mixture, because of the variation of nucleotide positions, contains randomly distributed sequence combinations which result in different translation products upon expression.

(5.) DNA vaccine according to paragraph (4.), characterized in that it contains a mixture of DNA molecules which code for the sequence variants of a viral protein or a part.

(6.) DNA vaccine according to paragraph (4.) characterized in that the mixture contains ≧10³ and preferably ≧10⁴ DNA molecules which differ from each other in their nucleic acid sequence.

(7.) DNA vaccine according to paragraph (4.), characterized in that it codes for a mixture of structurally different GP120 proteins of HIV, in which the vaccine contains a mixture of DNA molecules, the nucleic acid sequences of which differ from each other in the region coding for the V2 loop and/or in the region coding for the V3 loop.

(8.) DNA vaccine according to paragraph (7.), characterized in that it contains a mixture of DNA molecules which differ from each other in their nucleic acid sequence such that they code for a mixture of GP120 proteins which contain amino acid sequences which differ from each other in the V2 loop and/or in the V3 loop.

(9.) Nucleic acid sequence which is derived from the env sequence represented in SEQ ID NO: 1 or a fragment of same, characterized in that it is modified such that it contains monovalent restriction sites which make possible the specific exchange of the V2 and V3 regions.

(10.) Nucleic acid sequence which is derived from the env sequence represented in SEQ ID NO: 1 or a fragment of same, characterized in that it is modified such that it contains ten monovalent restriction cleavage sites at intervals of approx. 150 base pairs.

(11.) Nucleic acid sequence according to paragraph (9.) characterized in that the sequence is modified by the introduction of silent mutations.

(12.) Nucleic acid sequence according to paragraph (9.), characterized in that it contains the sequence given in SEQ ID NO: 9.

(13.) Nucleic acid sequence, characterized in that it contains the sequence given in SEQ ID NO: 11.

(14.) Nucleic acid sequence, characterized in that it contains the sequence given in SEQ ID NO: 12.

(15.) Single-stranded nucleic acid sequence which contains the region coding for the V3 loop and/or for the V2 loop of GP120, characterized in that in the V3 loop a 247 bp-long Bg1II-XbaI fragment or a 283 bp-long Bg1II-NheI fragment is exchanged for a modified fragment, and in the V2 loop a 139 bp-long PstI-Bc1I fragment or a 339 bp-long PstI-EcoRI fragment is exchanged for a modified fragment, the fragment/the fragments in each case containing inosine, a nucleic acid exchange or a mutation at at least 6 positions.

(16.) Single-stranded nucleic acid sequence which contains the region coding for the V2 loop and/or the region of GP120 coding for the V3 loop, characterized in that in the V3 loop a 247 bp-long Bg1II-XbaI fragment or a 283 bp-long Bg1II-NheI fragment is exchanged for a modified fragment, and in the V2 loop a 139 bp-long PstI-Bc1I fragment or a 339 bp-long PstI-EcoRI fragment is exchanged for a modified fragment, the fragment/the fragments in each case containing inosine, a nucleic acid exchange or a mutation at at least 6 positions, the nucleic acid sequence being complementary to the nucleic acid sequence according to paragraph (14.).

(17.) Nucleic acid sequence according to paragraph (15.) or a single-stranded nucleic acid sequence which contains the region coding for the V2 loop and/or the region of GP120 coding for the V3 loop, characterized in that in the V3 loop a 247 bp-long Bg1II-XbaI fragment or a 283 bp-long Bg1II-NheI fragment is exchanged for a modified fragment, and in the V2 loop a 139 bp-long PstI-Bc1I fragment or a 339 bp-long PstI-EcoRI fragment is exchanged for a modified fragment, the fragment/the fragments in each case containing inosine, a nucleic acid exchange or a mutation at at least 6 positions, the nucleic acid sequence being complementary to a nucleic acid sequence characterized in that it contains the sequence given in SEQ ID NO: 12, characterized in that the fragment/the fragments contain(s) inosine, a nucleic acid exchange or a mutation at 9 to 20 positions.

(18.) Double-stranded DNA which comprises hybrids of the single-stranded nucleic acid sequence according to paragraph (15.) with a single-stranded nucleic acid sequence which contains the region coding for the V2 loop and/or the region of GP120 coding for the V3 loop, characterized in that in the V3 loop a 247 bp-long Bg1II-XbaI fragment or a 283 bp-long Bg1II-NheI fragment is exchanged for a modified fragment, and in the V2 loop a 139 bp-long PstI-Bc1I fragment or a 339 bp-long PstI-EcoRI fragment is exchanged for a modified fragment, the fragment/the fragments in each case containing inosine, a nucleic acid exchange or a mutation at at least 6 positions, the nucleic acid sequence being complementary to a nucleic acid sequence characterized in that it contains the sequence given in SEQ ID NO: 12.

(19.) Nucleic acid mixture which comprises double-stranded DNAs, the nucleic acid sequences of which are derived from the env sequence in SEQ ID NO: 1 or SEQ ID NO: 11 or a fragment of same, characterized in that the nucleic acid sequences in each case differ from each other in the region coding for the V2 loop and/or in the region coding for the V3 loop.

(20.) Nucleic acid mixture according to paragraph (19.), characterized in that the nucleic acid sequences differ from each other such that they code for a mixture of proteins which in each case contain amino acid sequences which differ from each other in the V2 loop and/or in the V3 loop.

(21.) Nucleic acid mixture according to paragraph (20.), characterized in that the mixture contains ≧10² sequence variants.

(22.) Nucleic acid sequence according to paragraph (21.), characterized in that the mixture contains ≧10³ and preferably ≧10⁴ sequence variants.

(23.) Protein mixture which comprises sequence variants of the GP120 protein, characterized in that it is a mixture of proteins which contain amino acid sequences which in each case differ from each other in the V2 loop and/or in the V3 loop, the mixture being obtainable by expression of a plasmid DNA mixture which, because of the variation of nucleotide positions, contains randomly distributed sequence combinations.

(24.) Protein mixture according to paragraph (23.), characterized in that the mixture contains ≧10² sequence variants.

(25.) Protein mixture according to paragraph (24.), characterized in that the mixture contains ≧10³ and preferably ≧10⁴ sequence variants.

(26.) Plasmid, which contains an inserted double-stranded DNA according to paragraph (18.).

(27.) Expression vector, characterized in that it contains an inserted nucleic acid sequence according to paragraph (9.).

(28.) Expression vector according to paragraph (27.), characterized in that it contains the sequence given in SEQ ID NO: 10.

(29.) Expression vector, characterized in that it corresponds to DSM 12612.

(30.) Vector mixture which contains a mixture of plasmids according to paragraph (26.), characterized in that the nucleic acid sequences of the plasmids differ in each case from each other in the region coding for the V2 loop and/or in the region coding for the V3 loop, where the mixture of the plasmids, because of the variation of nucleotide positions, contains randomly distributed sequence combinations.

(31.) Vector mixture according to paragraph (30.), characterized in that the mixture contains ≧10² plasmids which differ from each other in their nucleic acid sequence.

(32.) Vector mixture according to paragraph (31.), characterized in that the mixture contains ≧10³ and preferably ≧10⁴ plasmids which differ from each other in their nucleic acid sequence.

(33.) Vector mixture according to paragraph (30.), characterized in that the plasmids can be expressed in E. coli as host cell.

(34.) Vector mixture according to paragraph (30.), characterized in that the plasmids can be expressed in eukaryotic cells, preferably in Cos, CHO or BHK cells, as host cells.

(35.) E. coli host cells which are transfected with a vector mixture according to paragraph (33.).

(36.) Eukaryotic host cells which are transfected with a vector mixture according to paragraph (34.).

(37.) Eukaryotic host cell according to paragraph (36.), characterized in that they are a host cell from the group consisting of Cos, BHK or CHO cells.

(38.) Process for the preparation of the nucleic acid sequence according to paragraph (10.), characterized in that so many silent mutations are introduced into a nucleic acid sequence coding for a viral protein that the nucleic acid sequence thereby obtained contains monovalent restriction sites which make possible the exchange of the V2 and V3 regions.

(39.) Process for the preparation of the nucleic acid sequence according to paragraph (10.), characterized in that so many silent mutations are introduced into a nucleic acid sequence coding for a viral protein that the nucleic acid sequence thereby obtained contains ten monovalent restriction sites at intervals of approx. 150 base pairs.

(40.) Process according to paragraph (38.) characterized in that the nucleic acid sequence coding for a viral protein is the sequence according SEQ ID NO: 1 or SEQ ID NO: 11 or a fragment of same.

(41.) Process for the preparation of the vector mixture according to paragraph (33.), characterized in that plasmids, the nucleic acid sequences of which in each case differ from each other in the region coding for the V2 loop and/or in the region coding for the V3 loop in each case through random distribution of the bases at the varied nucleotide positions, are ligated into a vector which can be expressed in host cells.

(42.) Process according to paragraph (41.), characterized in that the host cells are E. coli, Cos, CHO or BHK cells.

(43.) Process for the preparation of the host cells composing transforming E. coli. with a vector mixture according to paragraph (30.).

(44.) Process for the preparation of a protein vaccine which comprises a mixture of viral protein molecules, characterized in that the molecules are sequence variants of a single viral protein or of part of same, the mixture containing ≧10² sequence variants, which is obtainable by expression of a plasmid-DNA mixture which, because of the variation of nucleotide positions, contains randomly distributed sequence combinations, said process comprising cultivating host cells according to paragraph

(35.) under conditions which allow the expression of the mixture of viral protein sequence variants.

(45.) Process for the preparation of a DNA vaccine which codes for a mixture of structurally different virus proteins, characterized in that the vaccine contains a mixture of sequence variants of a viral DNA molecule or of part of same, the mixture containing ≧10² DNA molecules which differ from each other in their nucleic acid sequence, where the mixture, because of the variation of nucleotide positions, contains randomly distributed sequence combinations wherein said process is carried out according to paragraph (41.), wherein the plasmids are ligated into a vector which can be expressed in host cells of the organism to be vaccinated.

(46.) A method of preparing a vaccine comprising forming a mixture of structurally different viral proteins which are sequence variants of a viral protein or of part of same, for the prevention and/or therapy of a virus infection in humans.

(47.) A method of preparing a vaccine comprising forming a mixture according to paragraph (23.) for the prevention and/or therapy of a HIV infection in humans.

(48.) A method of preparing a vaccine comprising forming a mixture of DNA molecules which code for sequence variants of a viral protein or of part of same, for the prevention and/or therapy of a virus infection in humans.

(49.) A method of preparing a vaccine comprising forming a nucleic acid mixture according to paragraph (19.) for the prevention and/or therapy of a virus infection in humans.

(50.) A method of preparing a vaccine comprising forming the nucleic acid mixture according to paragraph (19.) for the expression in host cells selected from the group consisting of E. coli, Cos, CHO and BHK cells.

(51.) A method of producing a protein mixture which comprises sequence variants of the GP120 protein, characterized in that it is a mixture of proteins which contain amino acid sequences which in each case differ from each other in the V2 loop and/or in the V3 loop, the mixture being obtainable by expression of a plasmid DNA mixture which, because of the variation of nucleotide positions, contains randomly distributed sequence combinations; said method comprising expressing a vector mixture which contains a mixture of plasmids which contains an inserted double-stranded DNA which comprises hybrids of the single-stranded nucleic acid sequence according to paragraph (15.) with a single-stranded nucleic acid sequence which contains the region coding for the V2 loop and/or the region of GP120 coding for the V3 loop, characterized in that in the V3 loop a 247 bp-long Bg1II-XbaI fragment or a 283 bp-long Bg1II-NheI fragment is exchanged for a modified fragment, and in the V2 loop a 139 bp-long PstI-Bc1I fragment or a 339 bp-long PstI-EcoRI fragment is exchanged for a modified fragment, the fragment/the fragments in each case containing inosine, a nucleic acid exchange or a mutation at at least 6 positions, the nucleic acid sequence being complementary to a nucleic acid sequence characterized in that it contains the sequence given in SEQ ID NO: 12; characterized in that the nucleic acid sequences of the plasmids differ in each case from each other in the region coding for the V2 loop and/or in the region coding for the V3 loop, where the mixture of the plasmids, because of the variation of nucleotide positions, contains randomly distributed sequence combinations

(52.) A method of preparing a protein mixture which comprises sequence variants of the GP120 protein, characterized in that it is a mixture of proteins which contain amino acid sequences which in each case differ from each other in the V2 loop and/or in the V3 loop, the mixture being obtainable by expression of a plasmid DNA mixture which, because of the variation of nucleotide positions, contains randomly distributed sequence combinations, said method comprising culturing a host cell according to paragraph (35.).

(53.) Pharmaceutical composition for the prevention and/or therapy of a virus infection, characterized in that it comprises a protein mixture and a nucleic acid mixture, the protein mixture comprising sequence variants of a viral protein or of part of same, and the nucleic acid mixture comprising DNA molecules which code for sequence variants of a viral protein or of part of same.

(54.) Pharmaceutical composition for the prevention and/or therapy of a virus infection, characterized in that it comprises a protein mixture and a nucleic acid mixture, the protein mixture comprising sequence variants of a viral protein or of part of same, and the nucleic acid mixture comprising DNA molecules which code for sequence variants of a viral protein or of part of same, said composition comprising a protein mixture according to paragraph (23.) and a nucleic acid mixture which comprises double-stranded DNAS, the nucleic acid sequences of which are derived from the env sequence in SEQ ID NO: 1 or SEQ ID NO: 11 or a fragment of same, characterized in that the nucleic acid sequences in each case differ from each other in the region coding for the V2 loop and/or in the region coding for the V3 loop.

The present invention is explained in the following, using examples, figures and a sequence protocol.

### EXAMPLES

1. General Description of the Preparation Process
1.1 Cloning of V3 loop-coding oligonucleotides
1.2 Cloning of the V3 env variants
1.3 Analysis of variability

2. Material and Methods
2.1 Abbreviations
   2.1.1 General abbreviations
   2.1.2 Nucleic acids
2.2 Bacterial strains
2.3 Plasmids
2.4 Enzymes
2.5 Chemicals
2.6 Oligonucleotides
2.7 Molecular weight standards
2.8 Reagent kits used
2.9 Media
2.10 Sterilization of solutions
2.11 Cultivation and storage of bacteria
2.12 Preparation of competent cells
2.13 Transformation into E. coli
2.14 Plasmid-DNA preparation
2.15 Separation of DNA in agarose gels
2.16 Purification of DNA from agarose gels
2.17 Separation of DNA in polyacrylamide gels
2.18 Cleaving of DNA
2.19 Ligation of DNA
2.20 DNA sequencing
2.21 Preparation of double-stranded DNA with the help of oligonucleotides
2.22 Transfection of COS cells and CHO cells
2.23 Chromatographic purification of the GP120 mixture
2.24 Preparation of the DNA vaccine

1. General Description of the Preparation Process

1.1 Cloning of a Mixture of V3 Loop gp120 Variants

The oligonucleotides are synthesized as described under 2.6. As V3 loop sequence, a mutated sequence of the HIV-1 patient isolate F1-01 (M. Schreiber et al., J. Virol. 68 no. 6 (1994) 3908-3916) is given by way of example. Any other sequence or mixture of sequences is also possible. To clone many different variants of a V3 loop sequence, mixtures are used instead of pure nucleotide components at specific positions of the sequence. In this way, a mixture is obtained of oligonucleotides which all differ in the sequence and thus code for different V3 loops. Such oligonucleotide mixtures are also called oligonucleotides with degenerated sequences. Starting from chemically synthesized oligonucleotides with degenerated sequences, the region coding for the various V3 loops is shown.

An oligonucleotide, in reading-frame orientation (forward), is synthesized for the env-sequence section from the Bg1II cleavage site to the first degenerated position. A second oligonucleotide, in complementary orientation (reverse), is synthesized according to the sequence from a position which lies 15 bases before the beginning of the variable region (method 2.6). Through overlapping of the 3′ region along a length of 15 bases in total, the hybridization of both oligonucleotides is carried out. In a subsequent reaction with DNA polymerase (e.g. Taq DNA polymerase or Klenow fragment), a completely double-stranded DNA molecule forms from the two hybridized oligonucleotides (method 2.21).

The thus-obtained DNA mixture is digested with restriction endonucleases Bg1II and XbaI. The cloning of the DNA mixture is carried out in the expression vector ΔV3 pBSCenvV3 cleaved with Bg1II and XbaI (methods 2.15 and 2.16).

This vector contains the coding sequence of the gp160 of the HIV-1 strain NL4-3 (IIIB). The NL4-3 env gene was manipulated such that it possesses the restriction cleavage sites Bg1II and XbaI as well as ApaLI, PstI and Bc1I once each only. The region coding for the V3 loop, which lies between the cleavage sites Bg1II and XbaI, was removed and replaced by a 15 base pair sequence, whereby an analytical cleavage site was introduced for the enzyme AscI. The Bg1II- and SbaI-cleaved V3 loop DNA mixture is cloned into this vector (method 2.19). The loss of the AscI cleavage site in the finished V3 loop pBSCenvV3 vector can be used for the selection of the V3 loop coding clones (method 2.18).

The thus-formed plasmid mixture is transformed in DH5a bacteria (method 2.12), a transformation rate of >10⁵ having to be achieved. A gene library of V3 loop fragments with a size of approx. 10⁵ clones is thereby obtained. The mixture is to be analyzed by DNA sequencing (see 1.3). With the help of this process, a mixture of clones is obtained, which all code for different V3 loop variants of the GP120 protein. This mixture of vectors serves as starting product for the preparation of the protein mixture for use as inoculant and as starting product for use as DNA vaccine.

For the preparation of the GP120 protein mixture, the pBSCenvV3 expression vectors are transfected into COS cells and CHO cells (method 2.22). The purification of the different GP120 proteins, the active ingredient mixture, is carried out according to method 2.23, as described in the literature.

1.2 Cloning of a Mixture of V2 Loop gp120 Variants

Variants of the V1 loop and V2 loop are prepared in the same way. The expression vector AV3-pBSCenvV3 has three additional restriction cleavage sites for this. The variation of the V1 loop takes place through cloning into the ApaLI and PstI cleavage sites. The variation of the V2 loop takes place through cloning into the PstI and Bc1I cleavage sites.

1.3 Analysis of Variability

The analysis of the V3 loop mixtures takes place through DNA sequencing (method 2.20). The statistical selection of approx. 100-200 clones whose V1, V2 and V3 loop sequences are to be determined, is planned. If all these clones are different, the heterogeneity of the gene library and thus also the heterogeneity of the protein mixture can be measured with the help of a statistic calculation.

2. Material and Methods

2.1 Abbreviations

2.1.1 General abbreviations
μg microgram
μl microlitre

μmol micromole
APS ammonium persulphate
bp base pairs
dNTP desoxynucleoside triphosphate
DNA desoxyribonucleic acid
DTT dithiothreitol
EDTA ethylenediaminetetraacetic acid
g gram
GP glycoprotein
h hour
HIV human immunodeficiency virus
IPTG isopropyl-β-D-thiogalactopyranoside
MOPS 3-morpholinepropanesulfonic acid
OD optical density
PAGE polyacrylamide gel electrophoresis
PCR polymerase chain reaction
RT room temperature
TEMED N,N,N′,N′-tetramethylethylenediamine

2.1.2 Nucleic Acids

| | |
|---|---|
| A | adenine |
| C | cytosine |
| G | guanine |
| T | thymidine |

2.2 Bacterial Strains

Escherichia coli DH5α: F—, endA1, hsdr17, (rk-mk+), su-pE44, recA1, λ-, gyrA96, relA1, Φ80d lac z ΔM15

2.3 Plasmids

| | |
|---|---|
| pBSCenvATG | own design, the sequence is given in |
| | SEQ ID NO: 10. |

2.4 Enzymes

Restriction enzymes MBI-Fermentas, Gibco-BRL, Biolabs DNA polymerases MBI-Fermentas, Gibco-BRL T4-DNA ligase MBI-Fermentas

2.5 Chemicals

| | |
|---|---|
| [α-35S]dATP | Amersham Life Science |
| Agarose, ultra pure | GIBCO BRL |
| Ammonium persulphate | Merck |
| Ampicillin | US Biochemical |
| Bacto-Agar | Becton Dickinson |
| Bacto-Trypton | Becton Dickinson |
| Boric acid | Merck |
| Bromphenol blue | Merck |
| Calcium chloride | Merck |
| Desoxyribonucleotides | MBI-Fermentas |
| Dithiothreitol | Biotechnik, St. Leon-Rot |
| Glacial acetic acid | Merck |
| Ethidium bromide | Sigma |
| Ethylenediaminetetraacetic | Merck |
| acid | |
| Glycerine | Merck |
| Urea | ICN Biomedicals |
| Yeast extract | Becton Dickinson |
| Potassium chloride | Merck |
| Potassium dihydrogen | Merck |
| phosphate | |
| Magnesium chloride | Merck |
| Acrylamide mix | Roth |
| Sodium acetate | Merck |
| Sodium chloride | Merck |
| Disodium hydrogen phosphate | Merck |
| Sodium dihydrogen phosphate | Merck |
| 2-propanol | Merck |
| Sigmacote (chlorinated | Sigma |
| polysiloxane) | |
| N,N,N′,N′- | Merck |
| tetramethylethylenediamine | |
| Tris(hydroxymethyl)- | GIBCO BRL |
| aminomethane | |

2.6 Oligonucleotides

All oligonucleotides listed were prepared with the Expedite™ nucleic acid synthesis system of PE Biosystems (Weiterstadt). oligonucleotides are used for the cloning of env genes which differ from each other only in the sequence for the V3 loop. The sequences of the oligonucleotides are listed taking as an example the cloning of the V3 loop region for the HIV-1 patient isolate F1-01.

V3 loop: for cloning into pNL4-3/BglII-NheI,
F1-01, forward:

(SEQ ID NO: 3)

5′-AAG ATG TAG TAA TTA GAT CTG CCA ATT TCA CAG ACA
ATG
CTA AAA CCA TAA TAG TAC AGC TGA ACA CAT CGT TAG
AAA TTA
ATT GTA CAA GAC CCA ACA ACA AT ACA-3′
V3 loop: for cloning in pNL4-3/BglII-NheI, F1-01:

(SEQ ID NO: 4)

5′-TTT TGC TCT AGA AAT GTT ACA ATG TGC TTG TCT TAT
GTC
TCC TGT TGC AGC TTC TGT TGC ATG AAA TGC TCT CCC
TGG TCC
GAT ATG GAT ACT ATG- (GA) (AT) (GATC) TTT TCT TGT
ATT GTT
GTT GGG-3′
For sequencing of V3 loop clones, the oligonucle-
otides 7010, 7011 and the M13 standard primer were
used (M13, M13r).
Primer 7010:

(SEQ ID NO: 5)

5′-CCA TGT ACA AAT GTC AG-3′
Primer 7011:

(SEQ ID NO: 6)

5′-AAA ACT GTG CGT TAC AA-3′
M13-Primer:

(SEQ ID NO: 7)

5′-GTA AAA CGA CGG CCA GT-3′
M13r-Primer:

(SEQ ID NO: 8)

5′-CAG GAA ACA GCT ATG AC-3′

2.7 Molecular weight standards

| | |
|---|---|
| 1 kb conductor | MBI-Fermentas |
| 100 bp conductor | MBI Fermentas |

2.8 Reagent kits used

| | |
|---|---|
| T7 Sequencing-Kit | Pharmacia |
| Qiaquick PCR purification Kit | Qiagen |
| Qiagen Plasmid Kit | Qiagen |

2.9 Media
YT Medium:
   10 g trypton
5 g yeast extract
5 g NaCl

dYT medium:
16 g trypton
10 g yeast extract
5 g NaCl

dYT agar plates:
10 g trypton
5 g yeast extract
5 g NaCl

15 g agar

The quoted quantities relate in each case to 1000 ml deionized water. The mixtures were autoclaved at 121° C. and 1.5 bar for 20 mins. Should the media contain antibiotics or other heat-sensitive reagents, the corresponding quantities were sterile-filtered and added after cooling of the medium.

| | | |
|---|---|---|
| Ampicillin | 3.3 | ml/l (60 mg/ml) |
| IPTG | 3 | ml/l (100 mM) |
| xgal | 3 | ml/l (2% in DMF, do not filter) |

2.10 Sterilization of Solutions and Apparatus

Solutions and media as well as pipette tips, Eppendorf vessels and glass apparatus were autoclaved for 20-40 mins at 121° C. and 1.5 bar. Heat-sensitive solutions such as e.g. antibiotics and IPTG solutions were sterile-filtered.

2.11 Cultivation and Storage of Bacteria

Bacteria cultures were in each case inoculated with a single colony. To isolate a single colony, cells of a liquid culture were spread on an agar plate. After incubation at 37° C. overnight, isolated colonies have grown. For the short-term preservation of the bacteria, the agar plates were sealed with parafilm and stored at 4° C. For a long-term storage of bacteria strains, 0.75 ml of a YT-overnight culture were mixed with 0.25 ml sterile glycerine, shock-frozen in liquid nitrogen and stored at −70° C.

2.12 Preparation of Competent Cells

100 ml YT-medium were inoculated with 100 μl of an overnight culture. The bacteria were incubated until an OD₅₆₀=0.4 at 37° C. was reached in the shaking incubator and then centrifuged for 8 mins at 4° C. and 1000 g. All subsequent work was carried out on ice using pre-cooled vessels and 4° C. cold solutions. The cells were resuspended in 50 ml 50 mM CaCl₂ and incubated for 30 mins on ice. After renewed centrifuging, the bacteria were taken up in 2.5 ml sterile TFBII buffer and divided into portions of 100 μl each. The 100-μl portions of the competent cells were able to be, insofar as they were not required for an immediate transformation, stored in liquid nitrogen at −70° C. after shock-freezing.

TFBII buffer:

| 10 mM | MOPS pH 7.0 |
|---|---|
| 75 mM | CaCl₂ |
| 10 mM | KCl |
| 15% | glycerine |

2.13 Transformation of E. coli

(Hanahan, J. Mol. Biol. 166 (1983) 557-580) A 100 μl portion of competent cells was thawed in an ice bath and incubated with 1-100 ng plasmid-DNA for 30 mins at 0° C. The transformation mixture was then incubated for 1 min at 42° C. 1 ml YT-medium was then added and agitated for an hour at 37° C. To make possible a selection of the transformed cells, 100-500 μl of the mixture were spread onto antibiotic-containing YT agar plates. After incubation overnight at 37° C., only colonies which carry the plasmid-coded resistance gene have grown. Upon a transformation of cells which do not contain any functional β-galactosidase (lacZΔM15 mutation, e.g. DH5α), vectors such as the pUC used make possible a direct selection (blue white screening) of recombinant bacteria via a plasmid-coded β-galactosidase. For the blue-white selection, the bacteria were spread onto Amp/IPTG/Xgal-YT agar plates. The substrate Xgal is cleaved into a blue dye by the β-galactosidase. Because of the destruction of the reading frame of the lacZ gene by the insertion of a foreign DNA fragment into the lacZ gene, white colonies are produced. On the other hand, blue colonies mean that the lacZ gene has remained functional during cloning and ligation and no DNA was inserted.

2.14 Plasmid DNA Preparation

(Qiagen, Hilden)

To isolate plasmid-DNA from E. coli, the QIAprep Spin Miniprep Kit from Qiagen was used. The DNA preparation took place according to the manufacturer's instructions (QIAprep Plasmid Handbook March 1995). Plasmid-containing E. coli bacteria were inoculated in dYT-amp medium and agitated overnight at 37° C. Three ml of the E. coli overnight culture were used for the plasmid isolation. The bacteria were harvested (1 min, 14,000 rpm, Heraeus centrifuge) and taken up in 250 μl P1 buffer. The bacteria were decomposed through alkaline lysis (250 μl, 0.2N NaOH/1%-SDS). The mixture was neutralized through the addition of 3M potassium acetate, (350 μl, pH 5.5). The purification of the plasmid DNA is based on the selective bonding of plasmid DNA to DEAE anion exchanger columns. With the chosen salt concentrations and pH conditions, the plasmid DNA bonds to the DEAE matrix. The DEAE matrix was washed (750 μl, PE buffer, Qiagen). The plasmid DNA was then eluted with 50 μl H₂O and stored at −20° C.

To obtain plasmid DNA in a larger quantity (approx. 100 μg), 25 ml overnight culture were used. To purify the DNA, the QIAGEN Plasmid Midi Kit was used. The purification is based in this case on the principle described under the “QIAprep Spin Miniprep Kit” and was carried out according to the manufacturer's instructions (QIAGEN Plasmid Purification Handbook January 1997).

2.15 Separation of DNA in Agarose Gels

The separation of DNA took place through gel electrophoresis in agarose gels. Depending on the size of the fragments examined, 0.8-2% agarose were dissolved by boiling-up in TBE buffer (for analytical agarose gels) or TAE buffer (for preparative agarose gels). After cooling to approx. 60° C., the gel liquid was mixed with 1 μl ethidium bromide (10 mg/ml) and poured into a prepared gel bed with inserted comb. The completely cooled gel was coated in an electrophoresis chamber with TBE or TAE buffer and the comb removed. The DNA samples were mixed with ⅕ vol. coating buffer and pipetted into the sample bags. The fragments were separated at 5-10 volt/cm gel length for 0.5-2 h. For detection, the gel was photographed after the electrophoresis in UV transmitted light. The molecular weights of the DNA bands were measured compared with following DNA molecular weight markers.

TBE buffer:

| | |
|---|---|
| 100 mM | Tris |
| 100 mM | boric acid |
| 3 mM | EDTA |

TAE buffer:

| 40 mM | Tris |
|---|---|
| 2 mM | EDTA |
| 0.114% | glacial acetic acid |

Coating buffer:

| | |
|---|---|
| 0.25% | bromphenol blue |
| 0.25% | xylene cyanol |
| 30% | glycerine |
| 50 mM | EDTA |

2.16 Purification of DNA from Agarose Gels

For the extraction of the DNA from agarose gels, the “QIAquick Gel Extraction Kit” (Qiagen, Hilden) was used. The buffer conditions were selected such that the nucleic acids bond to the silica membrane of the columns, whilst low-molecular-weight bacteria components pass through the membrane (method 2.15). The process was carried out according to the “Qiaquick Gel Extraction Kit Protocol” of the QIAquick Spin Handbook July 1997. The purified DNA was in each case eluted from the silica membrane with 50 μl H₂O.

2.17 Separation of DNA in Polyacrylamide Gels

The separation of radioactively marked DNA fragments for the DNA sequencing was carried out using denaturizing polyacrylamide gels. Two purified glass plates were cleaned of fatty residues with ethanol and coated with 1 ml Sigmacote per plate to obtain a hydrophobic surface. Through the coating, a later tearing of the gel upon removal from the glass plate was to be avoided. Spacers were placed between the two glass plates, and served at the same time for lateral sealing. The whole apparatus was fixed with several clamps. To prepare the sequence gel, 21 g urea were dissolved in approx. 20 ml water. After the addition of 7.5 ml acrylamide mix and 5 ml 10× TBE buffer (see 2.15), the mixture was made up to 50 ml with water. The polymerization of the gel was started by the addition of 300 μl APS and 100 μl TEMED. Immediately afterwards, the gel solution was poured into the prepared gel apparatus and the pocket bottom shaped by inserting the flat back of the saw-tooth comb. The gel was stored horizontally at room temperature until polymerization was complete. After the insertion of the gel into the gel chamber, the latter was filled with TBE buffer. By rotating the saw-tooth comb, the deposit pockets were formed. A 20-min pre-run was carried out in order to heat the gel to the optimal operating temperature. The pockets were washed thoroughly with TBE buffer and then filled with 4-5 μl of the samples. The electrophoresis took place at 2 kV (150 watt) for approx. 2-3 h. The gel was removed from the gel chamber after the electrophoresis was ended and one of the glass plates raised carefully. By laying-on and light pressing, the gel was transferred onto Schleicher & Schuell paper (Whatman, England). After drying at 80° C. under vacuum, the gel was exposed on an X-ray film (Kodak BioMax MR-1, Sigma Deisenhofen) for 1-3 days at RT.

Acrylamide mix:
40% polyacrylamide
0.8% bisacrylamide

2.18 Cleaving of DNA

Restriction endonucleases recognize and hydrolyze enzyme-specific palindromic DNA sequences of mostly 4-8 nucleotides' length. Upon hydrolysis, depending on the type of enzyme, blunt ends or overhanging ends of single-stranded DNA form. For a restriction digestion, 0.1-60 μg DNA were incubated for 2-5 h at 37° C. with 2-120 units of a restriction enzyme in the buffer indicated by the manufacturer. The total volume was between 10 μl for analytical and 300 μl for preparative mixtures. For restrictions with two different enzymes, a buffer was chosen in which both enzymes have a sufficient activity, e.g. the EcoRI buffer for an EcoRI/BamHI digestion, or the conditions for the second enzyme were set after incubation with the first enzyme.

2.19 Ligation of DNA

DNA ligases catalyze the linking of DNA molecules, consuming NAD⁺ or ATP through formation of a phosphodiester bond between a free 5′ phosphate group and a 3′ hydroxyl group. A three- to five-times excess of DNA fragment was incubated overnight at 12° C. in ligation buffer with 100-500 ng cleaved plasmid and 5 units T4-DNA ligase as well as 10 nmol ATP. Only ligations of cohesive ends were carried out.

Ligation buffer:

| | |
|---|---|
| 40 mM | Tris-HCl pH 7.8 |
| 10 mM | MgCl₂ |
| 10 mM | DTT |
| 0.5 mM | ATP |

2.20 DNA Sequencing

(Sanger et al., Proc. Natl. Acad. Sci. USA 74 (1977) 5463-5467) The sequencing of DNA was carried out according to the chain-break method. Double-stranded plasmid DNA serves as substrate. Double-stranded DNA can be synthesized from single-stranded DNA starting from an oligonucleotide primer in the presence of dNTPs through a DNA polymerase. If a small proportion of didesoxynucleotides (ddNTPs) is contained in the nucleotide mixture, this leads to a randomly distributed incorporation of the ddNTPs, as the DNA polymerase cannot distinguish between dNTPs and ddNTPs. The incorporation leads to a breaking of the double-stranded synthesis, because of the absence of the 3′-hydroxyl group of the ddNTPs and, as it takes place statistically distributed, to DNA single strands of different lengths. As the synthesis mixture is divided in four and only one of the four ddNTPs is present in each sample, base-specific chain breaks result in the respective mixtures. To mark the synthesized single strand, α-³⁵S-dATP is added to the reaction. After denaturing and separation of the DNA via polyacrylamide gel electrophoresis, the bands can be autoradiographically detected and the sequence of the DNA strand read off direct.

The sequencings were carried out with the T7-Sequencing-Kit (Pharmacia) according to the manufacturer's instructions. Either the M13-Universal-Primer (Pharmacia) or Primer M13r was used as oligonucleotide primer. 32 μl (approx. 2 μg) of purified double-stranded plasmid DNA were denatured with 8 μl 2 M NaOH for 10 mins at RT. After the addition of 7 μl 3 M sodium acetate, pH 4.8, 4 μl H2O and 120 μl −20° ethanol, the DNA was precipitated for 20 mins at −70° C. The DNA was isolated by centrifuging (15 mins, 4° C.), washed twice with −2020 cold 70% ethanol and dried in a vacuum centrifuge. The DNA sediment was then resuspended in 10 μl H₂O and after the addition of 2 μl annealing buffer and 2 μl primer solution (10 pmol in water) hybridized with the oligonucleotide primer at 65° C. for 5 mins, 37° C. for 10 mins and 5 mins at RT. Directly afterwards, for the purpose of primer elongation and radioactive marking, 3 μl labelling mix, 1 μl α-³⁵S-DATP (1000 Ci/mmol, 10 μCi/μl) and 2 μl T7 polymerase solution (diluted 1:5 with enzyme dilution buffer) were added and incubated for 5 mins at RT. In each case, 4.5 μl of this mixture were placed onto a MicroSample Plate (Greiner) preheated to 37° C. in which 2.5 μl each of the four different dNTP/ddNTP mixes were present. After five minutes' incubation at 37° C., which served for further elongation and base-specific termination, the reactions were ended by the addition of 5 μl stop solution. Before application to the polyacrylamide gel, the samples were denatured for 2 mins at 80° C. The samples were stored at −20° C.

Annealing buffer:

| | | |
|---|---|---|
| 1 | M | Tris-HCl pH 7.6 |
| 100 | mM | MgCl₂ |
| 160 | mM | DTT |

Labelling mix:

| | |
|---|---|
| 1.375 | μM dCTP |
| 1.375 | μM dGTP |
| 1.375 | μM dTTP |
| 333.5 | mM NaCl |

Enzyme dilution buffer:

| | | |
|---|---|---|
| 20 | mM | Tris-HCl pH 7.5 |
| 5 | mM | DTT |
| 100 | μg/ml | BSA |

| | |
|---|---|
| 5% | glycerine |

Stop solution:

| 10 mM | EDTA |
|---|---|
| 97.5% | formamide |
| 0.3% | bromphenol blue |
| 0.3% | xylene cyanol |

| | | | | | |
|---|---|---|---|---|---|
| A-mix: | 840 | μM dCTP | C-Mix: | 840 | μM dATP |
| | 840 | μM dGTP | | 840 | μM dGTP |
| | 840 | μM dTTP | | 840 | μM dTTP |
| | 93.5 | μM dATP | | 93.5 | μM dCTP |
| | 14 | μM ddATP | | 14 | μM ddCTP |
| | 40 | mM Tris-HCl pH 7.6 | | 40 | mM Tris-HCl |
| | | | | | pH 7.6 |
| | 50 | mM NaCl | | 50 | mM NaCl |
| G-Mix: | 840 | μM dATP | T-Mix: | 840 | μM dATP |
| | 840 | μM dCTP | | 840 | μM dCTP |
| | 840 | μM dTTP | | 840 | μM dGTP |
| | 93.5 | μM dGTP | | 93.5 | μM dTTP |
| | 14 | μM ddGTP | | 14 | μM ddTTP |
| | 40 | mM Tris-HCl pH 7.6 | | 40 | mM Tris-HCl |
| | | | | | pH 7.6 |
| | 50 | mM NaCl | | 50 | mM NaCl |

2.21 Preparation of Double-Stranded DNA Using Oligonucleotides

The oligonucleotides were hybridized at 60° C. for the preparation of the DNA mixture. 100 pmol were used per nucleotide. 1 pmol of the hybridized sample was used for the PCR or 100 pmol for the Klenow reaction.

The following cycles were carried out for the PCR:

| | | |
|---|---|---|
| At the start: | 10 min | 94° C. |
| 30 cycles: | 1 min | 94° C. |
| | 1 min | 45° C. |
| | 1 min | 72° C. |
| At the end: | 10 min | 72° C. |

1 pmol hybridized oligonucleotide was used as DNA template for the PCR. PCR primers were used for all PCR reactions carried out in a final concentration of 0.1 pmol/μl. 1 unit of the Taq polymerase was used per 50 μl of the reaction mixture. The concentration of the dNTPs in the PCR mixture was set at 0.1 mM.

To prepare the DNA mixture with the help of the Klenow reaction, 100 pmol of the hybridized oligonucleotides was dissolved in Klenow buffer (50 mM Tris-HCl, pH 8.0; 50 mM MgCl₂, 10 mM DTT, 0.05 mM dNTP). The reaction was started by the addition of 5 units DNA polymerase I (Klenow fragment). After 30 min at 37° C., the reaction was stopped by heating to 75° C. (10 min).

2.22 Transfection of COS Cells and CHO Cells

5-10 μg linearized plasmid DNA are dissolved in 150 μl cell culture medium (without FKS and antibiotics). 30 μl of the transfection agent (SuperFect, Qiagen, Hilden) are added to the solution. The mixture is incubated for 10 min at RT and added to the cells after the addition of 1 ml medium. The COS cells (80% confluent growth) were cultivated in 60 mm plates and washed with PBS shortly before the transfection. After an incubation of 3 h at 37° C. and 5% CO₂, the transfection agent was removed. The cells were then washed 4 times with PBS and taken up in selection medium (DMEM, 5% FKS, 600 μg/ml Geneticin).

2.23 Chromatographic Purification of the GP120 Mixture

The chromatographic purification takes place according to standard methods (Techniques in HIV Research, Aldovini & Walker, Stockton Press, 1990; S. W. Pyle et al. Purification of 120,00 dalton envelope glycoprotein from culture fluids of human immunodeficiency virus (HIV)-infected H9 cells. AIDS Res Hum Retroviruses 1987, 3: 387-400). Cell extracts and cell culture overhangs of GP120-expressing COS cells are used for the isolation of the GP120 mixture. After centrifuging (25,000 g), the lysate is purified as follows:
1. Gel filtration (Sephadex G-20)
2. Immunoaffinity chromatography (anti-GP120 antibodies bound to CH-sepharose 4B)
3. Concentration through protein precipitation
4. Concentration through dialysis

2.24 Preparation of the DNA Vaccine

Starting material for the preparation of the DNA vaccine is the mixture of the BstEII-BamHI DNA fragments of the env gene which were used for the preparation of the GP120 protein mixture. A eukaryotic expression vector for the HIV-1 GP120 which is approved for DNA vaccinations (cf. e.g. J. D. Boyer et al., J Infect Dis 1997, 176: 1501-1509; J. D. Boyer et al., Nat Med 1997, 3: 526-532; M. L. Bagarazzi et al., J Med Primatol 1997, 26: 27-33) is modified such that in the env gene the cleavage sites for BstEII and BamHI are located at identical points of the gp120 reading frame. The mutated, variable env-gene fragments (BstEII-BamHI) are cloned into such a vector. The modification of the env genes in the V2 loop and V3 loop regions and their cloning into the DNA vaccine vector takes place analogously to the cloning steps which are carried out for the preparation of the gp120 mixture.

I claim:

1. DNA vaccine which codes for a mixture of structurally different virus proteins, characterized in that the vaccine contains a mixture of sequence variants of a viral DNA molecule or of part of same, the mixture containing ≧10² DNA molecules which differ from each other in their nucleic acid sequence, where the mixture, because of the variation of nucleotide positions, contains randomly distributed sequence combinations which result in different translation products upon expression.

2. DNA vaccine according to claim 1, characterized in that it contains a mixture of DNA molecules which code for the sequence variants of a viral protein or a part.

3. DNA vaccine according to claim 1 , characterized in that the mixture contains ≧10³ and preferably ≧10⁴ DNA molecules which differ from each other in their nucleic acid sequence.

4. DNA vaccine according to claim 1, characterized in that it codes for a mixture of structurally different GP120 proteins of HIV, in which the vaccine contains a mixture of DNA molecules, the nucleic acid sequences of which differ from each other in the region coding for the V2 loop and/or in the region coding for the V3 loop.

5. DNA vaccine according to claim 4, characterized in that it contains a mixture of DNA molecules which differ from each other in their nucleic acid sequence such that they code for a mixture of GP120 proteins which contain amino acid sequences which differ from each other in the V2 loop and/or in the V3 loop.

6. A method of preparing a vaccine comprising forming a nucleic acid mixture which comprises double-stranded DNAs, the nucleic acid sequences of which are derived from the env sequence in SEQ ID NO: 1 or SEQ ID NO: 11 or a fragment of same, characterized in that the nucleic acid sequences in each case differ from each other in the region coding for the V2 loop and/or in the region coding for the V3 loop, for the prevention and/or therapy of a virus infection in humans.

7. A method of preparing a vaccine comprising forming the nucleic acid mixture according to claim 6 for the expression in host cells selected from the group consisting of E. Coli, Cos, CHO and BHK cells.


SEQUENCE LISTING (27 sequences)

SEQ ID NO: 1 - DNA, 9709 bp, Human immunodeficiency virus
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggctgcacgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - protein, 854 aa, Human immunodeficiency virus
  Envelope Polyprotein
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGCTSMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 3 - DNA, 107 bp, Artificial Sequence
  Description of the artificial sequence oligonucleotide for cloning
aagatgtagtaattagatctgccaatttcacagacaatgctaaaaccataatagtacagctgaacacatcgttagaaattaattgtacaagacccaacaacaataca

SEQ ID NO: 4 - DNA, 120 bp, Artificial Sequence
  Description of the artificial sequence oligonucleotide for cloning
ttttgctctagaaatgttacaatgtgcttgtcttatgtctcctgttgcagcttctgttgcatgaaatgctctccctggtccgatatggatactatgrwnttttcttgtattgttgttggg

SEQ ID NO: 5 - DNA, 17 bp, Artificial Sequence
  Description of the artificial sequence sequencing primer
ccatgtacaaatgtcag

SEQ ID NO: 6 - DNA, 17 bp, Artificial Sequence
  Description of the artificial sequence sequencing primer
aaaactgtgcgttacaa

SEQ ID NO: 7 - DNA, 17 bp, Artificial Sequence
  Description of the artificial sequence sequencing primer
gtaaaacgacggccagt

SEQ ID NO: 8 - DNA, 17 bp, Artificial Sequence
  Description of the artificial sequence sequencing primer
caggaaacagctatgac

SEQ ID NO: 9 - DNA, 2148 bp, Artificial Sequence
  Description of the artificial sequence synthetic DNA
tgggtcaccgtctattatggggtgcctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagccttaagccatgtgtaaaattaaccccactctgtgttagtttaaagtgcactgatttgaagaatgatactaataccaatagtagtagcgggagaatgataatggagaaaggagagataaaaaactgcagcttcaatatcagcacaagcataagagataaggtgcagaaagaatatgcattcttttataaacttgatatagtaccaatagataataccagctataggttgataagttgtaacacctcagtgatcacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattcgaccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacatttctagagcaaaatggaatgccactttaaaacagatagctagcaaattaagagaacaatttggaaataataaaacaataatctttaagcagtcatccggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggccaaattagatgttcatcaaatattactgggctgctattaactcgagatggtggtaataacaacaatgggtccgagattttcagacctggaggaggcgatatgagggataattggagaagtgaattatataaatataaagtagtaaaaattgaaccattaggagtagcacccaccaaggcaaagagacgcgtggtgcagagagaaaagcgcgcagtgggaataggagctctgttccttgggttcttgggagcagcaggaagcactatgggcgcagcgtcaatgacgctgacggtacaggccagacaattattgtctgatatagtgcagcagcagaacaatttgctgagggcaattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatcc

SEQ ID NO: 10 - DNA, 6229 bp, Artificial Sequence
  Description of the artificial sequence synthetic DNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggacaattcgagctcggtaccgtcgacgccaccatgagagtgaaggagaagtatcagcacttgtggagatgggggtggaaatggggcaccatgctccttgggatattgatgatctgtagtgctacagaaaaattgtgggtcaccgtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggctgcacgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagatctagactagaactagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 860 bp, Human immunodeficiency virus
  misc_feature (1)..(860): PI-932 original sequence V1-V2-V3-loop
tgtgtacccacagaccccaacccacaaaaggtagtattggaaaatgtgacagaaaattttaacatgtggaaaaatgacatggtagaacagatgcatgaggatataatcaatttatgggatcaaagcctaaagccatgtgtaaaactaaccccactctgtgttactttaaattgcactgatgctgatttaaattgcaataatactgatttaaattgcactaaagctaatttggggaaaaatactcataacaatactattagtgggaaaataatagagaaagtagaaataaaaaactgctctttcaaggtcaccacaggcataagggataagatgcaaaaagaatatgcacttttgaataaacttgatatagtaccaatagataatgataagaataatactaactttatattgataagttgtaacacctcgaccattacacaggcctgtccaaaggtatcctttgagccaattcccatacatttttgtgccccggctggttttgcgattctaaagtgtaatgaaaagagttacagtggaaaaggaccatgtaaaaatgtcagcacagtacaatgtacacatggaattaggccagtagtgtcaactcaactgctgttgaatggcagtctagcagaaaaagaagtagtaattagatctgagaatttcacagacaatgctaaaaccataatagtacagctgaaggaatctgtaaacattacttgtataagaccccacaacactgtaacagacaggatacatatagggccagggagatcatttcatacaacaagaaaaataaaaggagatataagacaagcacattgtagccttaggagaaaagattggaataacactttacaaga

SEQ ID NO: 12 - DNA, 870 bp, Artificial Sequence
  Description of the artificial sequence PI-932 gene cassette, comprising the cleavage sites for restriction enzymes BspT1, PstI, BclI, EcoRI, BglII, PvuII, XbaII, NheI
tgtgtacccacagaccccaacccacaaaaggtagtattggaaaatgtgacagaaaattttaacatgtggaaaaatgacatggtagaacagatgcatgaggatataatcaatttatgggatcaaagccttaagccatgtgtaaaactaaccccactctgtgttactttaaattgcactgatgctgatttaaattgcaataatactgatttaaattgcactaaagctaatttggggaaaaatactcataactgcagtattagtgggaaaataatagagaaagtagaaataaaaaactgctctttcaaggtcaccacaggcataagggataagatgcaaaaagaatatgcacttttgaataaacttgatatagtaccaatagataatgataagaataatactaactttatattgataagttgtaacacctcggtgatcacacaggcctgtccaaaggtatcctttgagccaattcccatacatttttgtgccccggctggttttgcgattctaaagtgtaatgaaaagagttacagtggaaaaggaccatgtaaaaatgtcagcacagtacaatgtacacatggaattcggccagtagtgtcaactcaactgctgttgaatggcagtctagcagaaaaagaagtagtaattagatctgagaatttcacagacaatgctaaaaccataatagtacagctgaaggaatctgtaaacattacttgtataagaccccacaacactgtaacagacaggatacatatagggccagggagatcatttcatacaacaagaaaaataaaaggagatataagacaagcacattgtagcctttctagaaaagattggaataacactttacaagagatagctagc

SEQ ID NO: 13 - protein, 35 aa, Artificial Sequence
  Description of Artificial Sequence V3 loop sequence of HIV-1 patient isolate
CTRPNNNTRKSIHIGPGRAFYATGDIIGDIRQAHC

SEQ ID NO: 14 - protein, 35 aa, Artificial Sequence
  Description of Artificial Sequence PI-903 isolate
CTRPNNNTRGSIHIGPGSTNYATGAIIGDISQAHC

SEQ ID NO: 15 - protein, 35 aa, Artificial Sequence
  Description of Artificial Sequence PI-951 isolate
CTRPHNNTRKSINIGPGRAWYTTGDIIGDIRQAHC

SEQ ID NO: 16 - protein, 35 aa, Artificial Sequence
  Description of Artificial Sequence PI-918 isolate
CTRPSNNTRKSIHIGPGRAFYATGEIIGDIRQAHC

SEQ ID NO: 17 - protein, 35 aa, Artificial Sequence; identical to SEQ ID NO: 16
  Description of Artificial Sequence PI-970 ISOLATE

SEQ ID NO: 18 - protein, 35 aa, Artificial Sequence
  Description of Artificial Sequence PI-990 ISOLATE
CTRPNNNTRRSIPIGPGRAFYTTGDIVGDIRQAHC

SEQ ID NO: 19 - protein, 35 aa, Artificial Sequence
  Description of Artificial Sequence PI-991 ISOLATE
CTRPNNNTRKSIPIAPGRAFYATGEIIGNIRQAHC

SEQ ID NO: 20 - protein, 35 aa, Artificial Sequence
  Description of Artificial Sequence PI-952 ISOLATE
CIRPNNNTRKRITLGPGRVLYTTGEIIGDIRKAHC

SEQ ID NO: 21 - protein, 35 aa, Artificial Sequence
  Description of Artificial Sequence PI-93 ISOLATE
CIRPHNTVTDRIHIGPGRSFHTTRKIKGDIRQAHC

SEQ ID NO: 22 - protein, 35 aa, Artificial Sequence
  Description of Artificial Sequence PI-910 ISOLATE
CTRPSIQKRRSVRIGPGRSFIAARAATGDIRKAQC

SEQ ID NO: 23 - protein, 35 aa, Artificial Sequence
  Description of Artificial Sequence PI-911 ISOLATE
CTRPSIQKRRSVRIGPGRSFIATRAATGDIRKAQC

SEQ ID NO: 24 - protein, 35 aa, Artificial Sequence
  Description of Artificial Sequence PI-930 ISOLATE
CTRPYRNAKHRIMHIGPGRAFYATNVKGNIKQAHC

SEQ ID NO: 25 - protein, 51 aa, Artificial Sequence
  Description of Artificial Sequence NL4-3 V2-Loop
GEIKNCSFNISTSIRDKVQKEYAFFYKLDIVPIDNTSYRLISCNTSVITQA

SEQ ID NO: 26 - protein, 59 aa, Artificial Sequence
  Description of Artificial Sequence NL4-3 V3-Loop
VQLNTSVEINCTRPNNNTRKSIRIQRGPGRAFVTIGKIGNMRQAHCNISRAKWNATLKQ

SEQ ID NO: 27 - protein, 37 aa, HIV-1
  sequence variation in position 1 (1)..(1): Xaa can be N, T, H, D, K, S, E, I, Y, or can be absent
XCXXXXXXXXXXXXXXXXXXXXXXXXXXXXXXXXXXX